(19) United States
(12) Patent Application Publication
(10) Pub. No.: US 2018/0256509 A1
(43) Pub. Date: Sep. 13, 2018

(54) Nanoparticle Compositions and Methods Thereof to Restore Vascular Integrity

(71) Applicants: ALBERT EINSTEIN COLLEGE OF MEDICINE, INC., Bronx, NY (US); UNIVERSITY OF CALIFORNIA SAN DIEGO, LaJolla, CA (US)

(72) Inventors: Joel M. Friedman, South Orange, NJ (US); Mahantesh Navati, Bronx, NY (US); Adam J. Friedman, New York, NY (US); Pedro Cabrales, La Mesa, CA (US)

(73) Assignees: ALBERT EINSTEIN COLLEGE OF MEDICINE, INC., Bronx, NY (US); UNIVERSITY OF CALIFORNIA SAN DIEGO, La Jolla, CA (US)

(21) Appl. No.: 15/534,389

(22) PCT Filed: Nov. 3, 2015
(86) PCT No.: PCT/US2015/063751

§ 371 Date: Jun. 8, 2017

Related U.S. Application Data
(60) Provisional application No. 62/090,140, filed on Dec. 10, 2014.

(51) Int. Cl. A61K 9/51; A61K 31/12; A61K 33/04; A61K 45/06; A61P 29/00

(52) U.S. Cl. CPC A61K 9/5161; A61K 9/5146; A61K 9/5123; A61K 9/0019; A61K 33/04; A61K 45/06; A61P 29/00; A61K 31/12

(57) ABSTRACT

The present invention relates to methods for treating systemic inflammation. In certain embodiments, the method comprises administering a therapeutically effective amount of curcumin-selenium loaded nanoparticles. The curcumin-selenium loaded nanoparticles can comprise a matrix of chitosan, polyethylene glycol and tetramethoxysilane encapsulating curcumin and selenium. In certain embodiments, the method comprises administering a therapeutically effective amount of nitric oxide-releasing nanoparticles. The nitric oxide-releasing nanoparticles can comprise a matrix of chitosan encapsulating nitric oxide. In certain embodiments, the systemic inflammation can be caused by endotoxemia. In certain embodiments, the systemic inflammation can be caused by a Filovirus, including an Ebola virus or a Marburg virus. In certain embodiments, the methods for treating systemic inflammation in a subject result in the reduction of proinflammatory cytokines in a subject. In certain embodiments, the method of treatment is a combination therapy. The present invention also relates to methods of making compositions comprising nanoparticles.

### RELATED APPLICATIONS

This application claims the benefit of U.S. provisional application Ser. No. 62/090,140, filed Dec. 10, 2014 which is hereby incorporated by reference in its entirety.

### 1. INTRODUCTION

The present application relates to methods for treating and/or reversing systemic inflammation. In certain embodiments, the methods of the present application comprise administering a therapeutically effective amount of curcumin-selenium loaded nanoparticles. The curcumin-selenium loaded nanoparticles can comprise a matrix of chitosan, polyethylene glycol, and tetramethoxysilane, such that the matrix encapsulates curcumin and selenium. In certain embodiments of the present application, the methods comprise administering a therapeutically effective amount of nitric oxide-releasing nanoparticles. The nitric oxide-releasing nanoparticles can comprise a matrix of chitosan encapsulating nitric oxide. In certain embodiments, the systemic inflammation can be caused by endotoxemia. In certain embodiments, the systemic inflammation can be caused by a filovirus, including an Ebola virus or a Marburg virus. In certain embodiments, the methods for treating systemic inflammation in a subject result in the reduction of proinflammatory cytokines in a subject. In certain embodiments, the method of treatment is a combination therapy. In certain embodiments, the present application also relates to methods of making compositions comprising nitric oxide-releasing nanoparticles or curcumin-selenium loaded nanoparticles.

### 2. BACKGROUND

The properties of blood vessels are adversely affected in many disease states, including: conditions such as: cardiovascular disease, neurological conditions (including various brain cancers such as glioblastoma, as well as neurodegenerative diseases such as Alzheimer's), diabetes, edema, and cancers (especially metastases).

Many types of infections can also lead to edema, shock, or sepsis, which are also mediated by leaky vessels. Causative agents include gram positive bacteria, gram negative bacteria, anaerobic bacteria, fungal infections, and atypical bacteria.

In particular, Filoviruses (e.g., Ebola virus (EBOV) and Marburg virus (MARV)) are among the most lethal and destructive viruses. They cause severe, often fatal viral hemorrhagic fevers in humans and nonhuman primates (e.g., monkeys, gorillas, and chimpanzees). Filoviruses are of particular concern as possible biological weapons since they have the potential for aerosol dissemination and weaponization. Filoviridae are a family of RNA viruses. Two members of the Filoviridae family have been identified: EBOV and MARV. There is one identified strain of MARV and four identified subtypes (i.e., strains) of EBOV: Ebola-Zaire, Ebola-Sudan, Ebola-Ivory Coast (i.e., Ebola-Tai), and Ebola-Reston. The exact origin, locations, and natural habitat of Filoviridae are unknown. However, on the basis of available evidence and the nature of similar viruses, it is postulated that Filoviridae are zoonotic (i.e., animal-borne) and are normally maintained in an animal host that is native to the African continent.

For more than 30 years, EBOV has been associated with periodic episodes of hemorrhagic fever in Central Africa that produce severe disease in infected patients, with a massive inflammatory response occurring several days after the onset of the first symptoms. This inflammatory phase causes extensive “leakiness” in the vasculature of the infected individual leading to extensive leakage of bodily fluids out of the blood vessels. Mortality rates in outbreaks have ranged from 50% for the Sudan species of EBOV (SEBOV) to up to 90% for the Zaire species of EBOV (ZEBOV) (Sanchez et al., Filoviridae: Marburg and Ebola Viruses, in Fields Virology (eds. Knipe, D. M. & Howley, P. M.) 1409-1448 (Lippincott Williams & Wilkins, Philadelphia)). An outbreak late in 2007 caused by an apparently new species of EBOV in Uganda resulted in a fatality rate of about 25% (Towner et al., PLoS Pathog., 4:e1000212 (2008)). ZEBOV has also decimated populations of wild apes in this same region of Africa (Walsh et al., Nature, 422:611-614 (2003)).

Prevention and treatment of EBOV infections presents many challenges. In fact, there are no approved vaccines or postexposure treatment modalities available for preventing or managing EBOV infections. Patients instead receive supportive therapy, i.e., electrolyte and fluid balancing, oxygen, blood pressure maintenance, and treatment for any secondary infections.

Thus, there is a need for compositions and methods for treating and preventing EBOV infections.

The high mortality rate from EBOV arises from the very high infectivity of the EBOV resulting in an overwhelming of the host defense system. The consequence is a massive inflammatory response that causes a “cytokine storm” within the vasculature that undermines the integrity and normal functioning of the endothelial lining of blood vessels. The resultant condition is one of massive shock and vascular collapse including extreme leakiness of the blood vessels. Treatment of EBOV subsequent to being infected requires targeting these two major factors that directly contribute to the extremely high rate of mortality. At this time there are no easily deployed or cost effective therapies that address either the high rate of infectivity or the lethal vascular collapse.

Nitric oxide (NO) is a lipophilic, diatomic, free radical which is surprisingly stable and soluble in aqueous solutions when compared to other radical species (Zacharia and Deen, 2005). Endogenous NO is produced enzymatically via L-arginine conversion to NO by three distinct NO synthase (NOS) pathways (Moncada S. et al. 1991). With respect to vascular function, shear stress exerted on the luminal (endothelial) surface stimulates vascular endothelial NOS (eNOS), regulating numerous vascular functions, principally smooth muscle tension (Busse and Fleming 1998). Under normal physiological conditions, intravascular NO supplementation has many complications as well as is short lived due to the rapid scavenging rate of NO by hemoglobin within erythrocytes (Lancaster J. R., Jr. 1997). Thus, the current limitations of nitric oxide (NO) delivery systems have created a need for development of compounds that generate NO in a controlled and sustained manner.

Currently, the clinical therapeutic potential of NO has only been exploited via inhaled NO gas from pressurized tanks. While this approach is inconvenient and costly (Ichinose et al. 2004), inhaled NO gas is still the preferred and only approved NO treatment for acute pulmonary hypertension (Zapol W. M. 1996). Other alternatives for intravascular NO therapy include formulations based on compounds containing either NO or an NO precursor in a stable form, which typically lack the capacity for controlled and sustained delivery (Homer and Wanstall 1998). Thus, despite the considerable therapeutic potential of NO, systemic deployment of NO to the bedside has proven very difficult.

As such, there is an urgent need for effective treatments for preventing and reversing the potentially lethal vasculature leakage that occurs in conditions whose morbidity and mortality are driven by vascular leakage.

### 3. SUMMARY

According to a first aspect, a method of treating systemic inflammation in a subject is provided, the method comprising administering a therapeutically effective amount of curcumin-selenium loaded nanoparticles to the subject. In one aspect, the method is for the early treatment of systemic inflammation wherein the curcumin-selenium loaded nanoparticles are administered to the subject within 24 hours of the onset of systemic inflammation. In another aspect, the curcumin-selenium loaded nanoparticles are administered to the subject prior to onset of symptoms of systemic inflammation. In one aspect, systemic inflammation is characterized by an increase in cytokines. The curcumin-selenium loaded nanoparticles can comprise a matrix of chitosan, polyethylene glycol (PEG) and tetramethoxysilane (TMOS) encapsulating the curcumin and selenium. In one aspect, the systemic inflammation in the subject is caused by endotoxemia. According to another aspect, the systemic inflammation in the subject is caused by a Filovirus, such as an Ebola virus or a Marburg virus.

According to another aspect, the curcumin-selenium loaded nanoparticles are administered in combination with one or more antiviral treatments. According to one aspect, the curcumin-selenium loaded nanoparticles are administered in combination with one or more anti-inflammatory treatments. According to a further aspect, the curcumin-selenium loaded nanoparticles are administered in combination with one or more additional therapeutic agents.

According to one aspect, the administering of the curcumin-selenium loaded nanoparticles results in an increased heart rate in the subject. According to another aspect, the administering of the curcumin-selenium loaded nanoparticles results in decreased arteriolar diameter in the subject. According to another aspect, the administering of the curcumin-selenium loaded nanoparticles results in increased arteriolar blood flow in the subject.

According to another aspect, the administering of the curcumin-selenium loaded nanoparticles results in increased functional capillary density in the subject.

According to one aspect, the administering of the curcumin-selenium loaded nanoparticles results in the reduction of one or more cytokines in the subject. According to another aspect, the administering of the curcumin-selenium loaded nanoparticles results in the reduction of one or more proinflammatory cytokines in the subject.

According to another aspect, the administering of the curcumin-selenium loaded nanoparticles results in a decrease in vascular permeability in the subject.

According to a further aspect, the curcumin-selenium loaded nanoparticles are administered by incorporation into an intravenous (IV) infusion, or by transmucosal systemic delivery.

According to another aspect, a method for reversing systemic inflammation in a subject is provided, the method comprising administering a therapeutically effective amount of nitric oxide-releasing nanoparticles to a subject. According to another aspect, the method is for the early treatment of systemic inflammation in a subject, the method comprising administering a therapeutically effective amount of nitric oxide-releasing nanoparticles to a subject within 48 hours of the onset of systemic inflammation. In one aspect, the nitric oxide-releasing nanoparticles are administered to a subject within 24 hours of the onset of systemic inflammation. In one aspect, the nitric oxide-releasing nanoparticles are administered to a subject within 12 hours of the onset of systemic inflammation. In one aspect, the nitric oxide-releasing nanoparticles are administered to a subject prior to an increase in cytokines associated with systemic inflammation. The nitric oxide-releasing nanoparticles can comprise a matrix of chitosan encapsulating the nitric oxide. According to a further aspect, the matrix of the nitric oxide-releasing nanoparticles further comprises PEG and TMOS. Alternatively, according to another aspect, the matrix of the nitric oxide-releasing nanoparticles further comprises PEG and TEOS.

According to one aspect, the administering of the nitric oxide-releasing nanoparticles results in the reduction of one or more cytokines in the subject. According to another aspect, the administering of the nitric oxide-releasing nanoparticles results in the reduction of one or more proinflammatory cytokines in the subject.

In one aspect, the systemic inflammation in the subject is caused by a Filovirus. According to a further aspect, the Filovirus is an Ebola virus or a Marburg virus.

According to another aspect, the nitric oxide-releasing nanoparticles are administered by incorporation into an intravenous (IV) infusion, or by transmucosal systemic delivery. In a further aspect, the transmucosal systemic delivery is by an inhaler, sublingual gel, rectal suppository, or a combination of both delivery methods.

According to one aspect, the nitric oxide-releasing nanoparticles are administered in combination with one or more antiviral treatments. According to another aspect, the nitric oxide-releasing nanoparticles are administered in combination with one or more anti-inflammatory treatments. According to another aspect, the nitric oxide-releasing nanoparticles are administered in combination with one or more chemotherapeutic agents, small organic molecules, cytotoxic agents, siRNA's, therapeutic antibodies, or any combination thereof.

According to another aspect, a method for reversing systemic inflammation in a subject is provided, the method comprising administering a therapeutically effective amount of nitric oxide-releasing nanoparticles to a subject, the nitric oxide-releasing nanoparticles comprising a matrix of trehalose and non-reducing sugar or starch encapsulating the nitric oxide. According to a further aspect, the matrix of the nitric oxide-releasing nanoparticles further comprises PEG and TMOS. Alternatively, according to another aspect, the matrix of the nitric oxide-releasing nanoparticles further comprises PEG and TEOS.

According to another aspect, a method of making a curcumin and selenium-loaded nanoparticle is provided, the method comprising: hydrolyzing TMOS by adding HCl to form a TMOS-HCl mixture; sonicating the TMOS-HCl mixture in an ice water bath; refrigerating the TMOS-HCl mixture until it is monophasic; dissolving a curcumin-selenium complex in methanol, wherein the curcumin-selenium complex comprises selenium tetrachloride and curcumin in a molar ratio of 1:4; combining the dissolved curcumin-selenium complex with chitosan, polyethylene glycol and the TMOS-HCl mixture under conditions of continuous sonication to form a polymerized gel; lyphilizing the polymerized gel to form a powder; and ball milling the powder.

According to another aspect, a method for alleviating vascular leakage in a subject is provided, the method comprising administering a therapeutically effective amount of a nitric oxide-releasing nanoparticle to a subject in need thereof.

According to another aspect, a method for restoring Nitric Oxide (NO) gradient in a subject suffering from shock or acute respiratory distress syndrome (ARDS) is provided, the method comprising administering a therapeutically effective amount of a nitric oxide-releasing nanoparticle.

According to another aspect, a method of delivering sustained release NO or S-nitrosothiols to a target location in a subject exhibiting vascular leakage is provided, the method comprising administering to the subject a therapeutically effective amount of a nitric oxide-releasing nanoparticle. According to a further aspect, the subject is exhibiting symptoms of a viral hemorrhagic fever. According to a further aspect, the subject is exhibiting symptoms of non-infectious conditions such as: cardiovascular disease, neurological conditions (including various brain cancers such as glioblastoma, as well as neurodegenerative diseases such as Alzheimer's), diabetes, edema, and cancers (especially metastases). According to another aspect, the subject is exhibiting symptoms of one or more infectious conditions/diseases such as sepsis or edema caused by bacterial, fungal, or viral agents, and in particular, various hemorrhagic fevers including Filoviral infections such as Ebola and Marburg virus infections. According to a further aspect, the nitric oxide-releasing nanoparticles are administered by incorporation into an intravenous (IV) infusion, or by transmucosal systemic delivery. In yet another aspect, the transmucosal systemic delivery is by sublingual gel, rectal suppository, or a combination of both delivery methods. In a further aspect, the nitric oxide-releasing nanoparticles are administered in combination with one or more palliative treatments, and/or one or more antiviral treatments. In yet another aspect, the nitric oxide-releasing nanoparticles are administered in combination with one or more chemotherapeutic agents, small organic molecules, cytotoxic agents, siRNA's, therapeutic antibodies, or any combination thereof.

### 3.1 Definitions

“Treating” or “treatment” of a state, disorder or condition includes:

(1) preventing or delaying the appearance of clinical symptoms of the state, disorder, or condition developing in a person who may be afflicted with or predisposed to the state, disorder or condition but does not yet experience or display clinical symptoms of the state, disorder or condition; or

(2) inhibiting the state, disorder or condition, i.e., arresting, reducing or delaying the development of the disease or a relapse thereof (in case of maintenance treatment) or at least one clinical symptom, sign, or test, thereof; or

(3) relieving the disease, i.e., causing regression of the state, disorder or condition or at least one of its clinical or sub-clinical symptoms or signs.

The benefit to a subject to be treated is either statistically significant or at least perceptible to the patient or to the physician.

An “immune response” refers to the development in the host of a cellular and/or antibody-mediated immune response to a composition or vaccine of interest. Such a response usually consists of the subject producing antibodies, B cells, helper T cells, suppressor T cells, regulatory T cells, and/or cytotoxic T cells directed specifically to an antigen or antigens included in the composition or vaccine of interest.

As used herein, the term “vaccine” refers to a composition comprising a cell or a cellular antigen, and optionally other pharmaceutically acceptable carriers, administered to stimulate an immune response in an animal, most preferably a human, specifically against the antigen and preferably to engender immunological memory that leads to mounting of a protective immune response should the subject encounter that antigen at some future time. Vaccines often include an adjuvant.

A “therapeutically effective amount” means the amount of a compound that, when administered to an animal for treating a state, disorder or condition, is sufficient to effect such treatment. The “therapeutically effective amount” will vary depending on the compound, the disease and its severity and the age, weight, physical condition and responsiveness of the animal to be treated.

The compositions of the invention may include a “therapeutically effective amount” or a “prophylactically effective amount” of a curcumin composition, a curcumin-seleniumn complex composition, a nitric oxide (NO) composition, a sustained release NO composition or a S-nitrosothiol composition as described herein. A “therapeutically effective amount” refers to an amount effective, at dosages and for periods of time necessary, to achieve the desired therapeutic result. A therapeutically effective amount of a compound of the present application may vary according to factors such as the disease state, age, sex, and weight of the individual, and the ability of the compound to elicit a desired response in the individual. A therapeutically effective amount is also one in which any toxic or detrimental effects of the compound are outweighed by the therapeutically beneficial effects A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

While it is possible to use a composition provided by the present invention for therapy as is, it may be preferable to administer it in a pharmaceutical formulation, e.g., in admixture with a suitable pharmaceutical excipient, diluent or carrier selected with regard to the intended route of administration and standard pharmaceutical practice. Accordingly, in one aspect, the present invention provides a pharmaceutical composition or formulation comprising at least one active composition, or a pharmaceutically acceptable derivative thereof, in association with a pharmaceutically acceptable excipient, diluent and/or carrier. The excipient, diluent and/or carrier should be “acceptable” in the sense of being compatible with the other ingredients of the formulation and not deleterious to the recipient thereof.

As used herein, the phrase “pharmaceutically acceptable” refers to molecular entities and compositions that are “generally regarded as safe”, e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopoeia or other generally recognized pharmacopeias for use in animals, and more particularly in humans.

“Patient” or “subject” refers to mammals and includes human and veterinary subjects.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, olive oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

As used herein, the term “adjuvant” refers to a compound or mixture that enhances the immune response to an antigen. An adjuvant can serve as a tissue depot that slowly releases the antigen and also as a lymphoid system activator that non-specifically enhances the immune response (Hood et al., Immunology, Second Ed., 1984, Benjamin/Cummings: Menlo Park. Calif., p. 384). Often, a primary challenge with an antigen alone, in the absence of an adjuvant, will fail to elicit a humoral or cellular immune response. Adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, and potentially useful human adjuvants such as N-acetyl-muramyl-L-threonyl-D-isoglutamine (thr-MDP), N-acetyl-nor-muramyl-L-alanyl-D-isoglutamine, N-acetylmuramyl-L-alanyl-D-isoglutaminyl-L-alanine-2-(1′-2′-dipalmitoyl-sn-glycero-3-hydroxyphosphoryloxy)-ethylamine, and BCG (bacille Calmette-Guerin). Preferably, the adjuvant is pharmaceutically acceptable.

### Abbreviations

NO nitric oxide
GSH glutathione
GSNO S-nitrosoglutathione
RSNO S-nitrosothiols containing molecules
NO-np nitric oxide releasing nanoparticles
SNO-np S-nitrosothiol loaded nanoparticles
NAC-SNO-np S-nitroso-N-acetylcysteine releasing nanoparticles
TMOS Tetramethoxysilane
TEOS Tetraethoxysilane
MPTS 3-mercaptopropyltrimethoxysilane
RPHPLC reverse phase high performance liquid chromatography
PBS phosphate buffered saline
DTPA diethylenetriaminepenta-acetic acid
MAP mean arterial blood pressure
HR heart rate
MetHb methemoglobin
BE base excess
LPS lipopolysaccharides
BL baseline

### 4. BRIEF DESCRIPTION OF THE FIGURES

FIG. 1 graphs show that administration of NO np (NO nanoparticles) early on after inoculation with LPS, prevents the progressive increase of inflammatory cytokines over time. Proinflammatory cytokines increased at a faster rate (TNFα, IL-1β and IL-6) than repair cytokine IL-10 without NO supplementation. Without NOnp treatment, the cytokine levels rise indicating the onset and progression of inflammation. NOnp treatment four hours after the LPS treatment results in a substantial reduction in the LPS induced increase in cytokine levels indicating that with NOnp treatment anti-inflammatory activity is present.

FIGS. 2A-B. FIG. 2A shows fluorescein isothiocyanate (FITC) microvessel staining that illustrates the rapid shedding of the glycocalyx during severe inflammation, in a time scale similar to the reduction of functional capillary density and decreased capillary flow. FIG. 2B shows the normalization to baseline (BL) amounts of glycocalyx degraded within 45 min after inoculation with LPS. Administration of NO nanoparticles prevented the rapid destruction of the glycocalyx.

FIGS. 3A-B. FIG. 3A shows varying intensity of FITC staining indicating vascular permeability. FITC labeled macromolecules should remain within the vascular compartment, but systemic inflammation increases vascular permeability and allows for the extravasation of macromolecules into the interstitial space (extravascular compartment). FIG. 3B shows the ratio of concentration of FITC labeled macromolecules between the intravascular (IV) and extravascular (EV) compartments at baseline (BL) and 60 min after inoculation with LPS. The normal ration of concentration of macromolecules between the IV and EV compartments is below 50% (0.5 ratio). Systemic inflammation increases that ratio to be above 1, where more macromolecules are present in the IV compartment compared to the EV compartment. Administration of NO nanoparticles reduced and slows down the changes in permeability resulting from the inflammation.

FIGS. 4A-B show bar graphs of the cytokine profiles (TNFα, TGFβ, MCP-1, IL-1α, IL-1β, IL-6, IL-10, IL-12) from the macrophages for C57BL mice (8 weeks old; 2.2 g) inoculated with 10 mg of LPS and then treated with either 10 mg/kg NO-NPs or 10 mg/kg control NPs 4 hours after LPS inoculation. Cytokine profiles were determined for both experimental groups (NO-NPs and control NPs) at 24 hours (FIG. 4A) and 48 hours (FIG. 4B).

FIGS. 5A-D show the flow cytometry results for C57BL mice (8 weeks old; 2.2 g) inoculated with 10 mg of LPS and then treated with either 10 mg/kg NO-NPs or 10 mg/kg control NPs 4 hours after LPS inoculation. Cells from animals from both experiment groups were incubated with anti-mouse CD14/CD163 (FIGS. 5A-B) and CD11c/CD206 (FIGS. 5C-D).

FIG. 6 shows the survival proportions for C57BL mice (8 weeks old; 2.2 g) inoculated with 10 mg of LPS and then treated with either 10 mg/kg NO-NPs or 10 mg/kg control NPs 4 hours after LPS inoculation.

FIGS. 7A-B show bar graphs of the cytokine profiles (TNFα, TGFβ, MCP-1, IL-1α, IL-1β, IL-6, IL-10, IL-12) for C57BL mice (8 weeks old; 2.2 g) inoculated with 10 mg of LPS and then treated with either 10 mg/kg NO-NPs or 10 mg/kg control NPs 24 hours after LPS inoculation. Cytokine profiles were determined for both the NO-NPs experimental group (FIG. 7A) and control NPs experimental group (FIG. 7B) at baseline, 24 hours and 48 hours after LPS inoculation.

FIG. 8 shows the survival proportions for C57BL mice (8 weeks old; 2.2 g) inoculated with 10 mg of LPS and then treated with either 10 mg/kg NO-NPs or 10 mg/kg control NPs 24 hours after LPS inoculation.

FIGS. 9A-E show bar graphs of the cytokine profiles (TNFα, TGFβ, MCP-1, IL-1α, IL-1β, IL-6, IL-10, IL-12) for C57BL mice infused with 10 mg/kg of LPS (Lipopolysaccharides from E. coli serotype 0128:B12, Sigma Aldrich St. Louis, Mo.), and then treated with 1 of 5 treatments: 1) no treatment (FIG. 9A); 2) control NP 10 mg/kg (FIG. 9B); 3) curcumin 10 mg/kg (FIG. 9C); 4) curcumin-NP 10 mg/kg (dose calculated based on curcumin concentration) (FIG. 9D); and 5) curcumin-selenium-NP 10 mg/kg (dose calculated based on curcumin concentration) (FIG. 9E). Cytokine profiles were determined for each treatment group at baseline, 1 hour, 6 hours, and 48 hours after LPS inoculation.

FIG. 10 shows a bar graph comparing the vascular permeability (intravascular-extravascular intensity) of C57BL mice infused with 10 mg/kg of LPS (Lipopolysaccharides from E. coli serotype 0128:B12, Sigma Aldrich St. Louis, Mo.), and then treated with 1 of 5 treatments: 1) no treatment; 2) control NP 10 mg/kg; 3) curcumin 10 mg/kg; 4) curcumin-NP 10 mg/kg (dose calculated based on curcumin concentration); and 5) curcumin-selenium-NP 10 mg/kg (dose calculated based on curcumin concentration). Vascular permeability was compared for all treatment groups at baseline and 2 hours after LPS infusion.

FIG. 11 shows a bar graph comparing the heart rates (HR) of C57BL mice infused with 10 mg/kg of LPS (Lipopolysaccharides from E. coli serotype 0128:B12, Sigma Aldrich St. Louis, Mo.), and then treated with 1 of 4 treatments: 1) untreated; 2) control-NP 10 mg/kg; 3) curcumin-NP 10 mg/kg (dose calculated based on curcumin concentration); and 4) curcumin-selenium-NP 10 mg/kg (dose calculated based on curcumin concentration). HR was compared for all treatment groups at baseline, and at 2, 6, 12, and 24 hours after LPS infusion.

FIGS. 12A-B show bar graphs comparing the microcirculation hemodynamics, specifically arteriolar diameter (FIG. 12A) and arteriolar blood flow (FIG. 12B), of C57BL mice infused with 10 mg/kg of LPS (Lipopolysaccharides from E. coli serotype 0128:B12, Sigma Aldrich St. Louis, Mo.), and then treated with 1 of 4 treatments: 1) untreated; 2) control-NP 10 mg/kg; 3) curcumin-NP 10 mg/kg (dose calculated based on curcumin concentration); and 4) curcumin-selenium-NP 10 mg/kg (dose calculated based on curcumin concentration). All treatment groups were evaluated for both hemodynamic end points at baseline, and at 2, 6, 12, and 24 hours after LPS infusion.

FIG. 13 shows a bar graph comparing the functional capillary density of C57BL mice infused with 10 mg/kg of LPS (Lipopolysaccharides from E. coli serotype 0128:B12, Sigma Aldrich St. Louis, Mo.), and then treated with 1 of 4 treatments: 1) untreated; 2) control-NP 10 mg/kg; 3) curcumin-NP 10 mg/kg (dose calculated based on curcumin concentration); and 4) curcumin-selenium-NP 10 mg/kg (dose calculated based on curcumin concentration). All treatment groups were compared at baseline, and at 2, 6, 12, and 24 hours after LPS infusion.

FIG. 14 shows a schematic diagram of an embodiment of the nanoparticle. A hybrid-hydrogel (silane-derived) nanoparticle with attached derivatized PEG of varying sizes in accordance with one or more embodiments is shown. The x on the PEG chain represents the specific derivative that allows for attachment of any of several possible cell and tissue-specific targeting molecules. PEG increases the circulation time, enhances crossing of the blood-brain barrier, limits aggregation, increases localization in tumors, and allows for attachment of a wide variety of molecules (as well as imaging). Also shown are spheres indicating encapsulated reagents, therapeutics, and/or imaging agents.

### 5. DETAILED DESCRIPTION

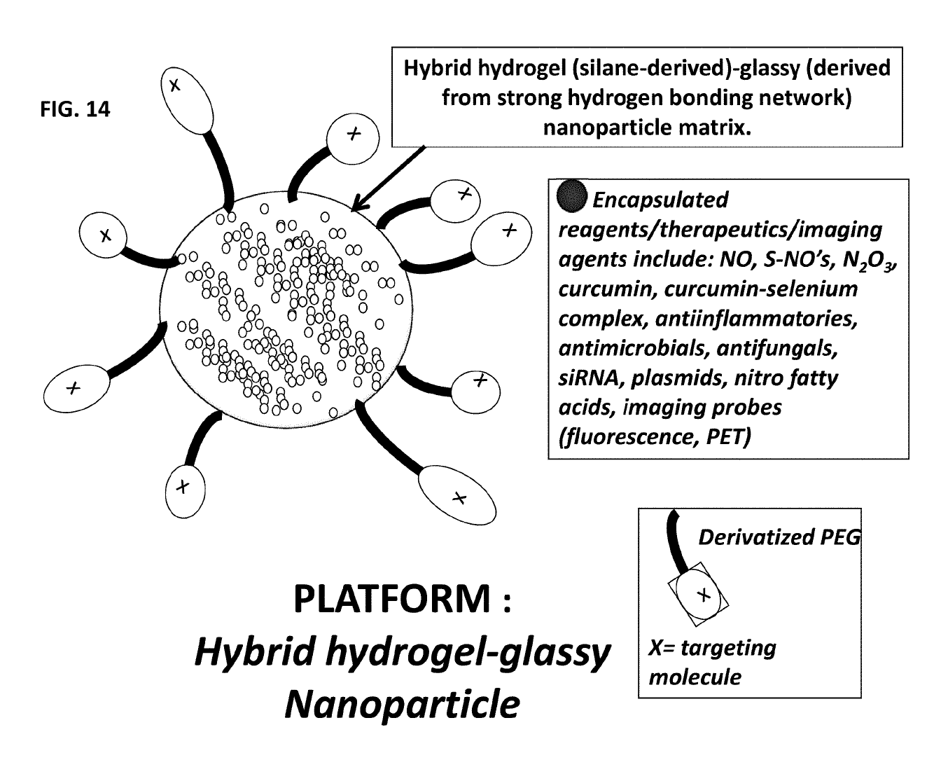

This application relates to nanoparticle delivery platform that allows for the systemic delivery of either curcumin alone (curcumin-loaded nanoparticles) or curcumin complexed with selenium (curcumin-selenium loaded nanoparticles). This application also relates to a nanoparticle deliver platform that allows for the systemic delivery of nitric oxide-releasing nanoparticles. In certain embodiments, the nanoparticles are hybrid-hydrogel nanoparticles (see FIG. 14). Treatment with curcumin-loaded (or curcumin-selenium loaded) nanoparticles and/or nitric oxide-releasing nanoparticles (generally referred to herein as “modified nanoparticles”) can ameliorate and/or reverse the cascade of inflammatory events resulting from lethal vascular collapse that are associated with many vascular-effecting ailments, such as Filovirus (e.g., Ebola virus) infections. These nanoparticle combinations are also proposed to reduce the viral load associated with Filovirus infections.

In the following detailed description, numerous specific details are set forth to provide a thorough understanding of the claimed subject matter. However, it will be understood by those skilled in the art that claimed subject matter may be practiced without these specific details. In other instances, methods, apparatuses, or systems that would be known by one of ordinary skill in the art have not been described in detail so as not to obscure claimed subject matter. It is to be understood that particular features, structures, or characteristics described may be combined in various ways in one or more implementations.

In general, disclosed herein are preparation and administration of modified nanoparticles and/or pharmaceutical compositions comprising modified nanoparticles. In one or more embodiments, methods of preparing modified nanoparticles and/or pharmaceutical compositions comprising modified nanoparticles are provided. In one or more embodiments, methods of treating, preventing or managing a disease or disorder in humans by administering a pharmaceutical composition comprising an amount of modified nanoparticles are provided. Also provided herein is a method of treatment comprising administering to the subject an effective amount of one or more of the nanoparticles disclosed herein and a pharmaceutically acceptable carrier. Further, provided herein is a pharmaceutical composition comprising any of the nanoparticles disclosed herein and a pharmaceutically acceptable carrier.

In one or more embodiments, the nanoparticles of the present application are hybrid hydrogel nanoparticles. The hybrid hydrogel nanoparticles include a hybrid-hydrogel (silane-derived)-glassy matrix (“matrix”), which is derived from a strong hydrogen bonding network. More specifically, the matrix can include, for example, at least one silane (e.g., TMOS, TEOS), as well as chitosan, polyethylene glycol (PEG), or polyvinyl alcohol (PVA), trehalose, and/or non-reducing sugar or starch, as well as other compounds as explained in greater detail in the methods disclosed below. FIG. 14 shows a schematic diagram of a hybrid-hydrogel (silane-derived) nanoparticle with attached derivatized PEG of varying sizes in accordance with one or more embodiments. The hybrid hydrogel nanoparticles can include specific derivatives (as shown by the “x” on the PEG chain) that allow for attachment of any of several possible cell and tissue-specific targeting molecules. PEG increases the circulation time, enhances crossing of the blood-brain barrier, limits aggregation, increases localization in tumors, and allows for attachment of a wide variety of molecules (as well as imaging). Also shown in FIG. 14 are spheres within the hybrid hydrogel nanoparticle matrix, indicating encapsulated reagents, therapeutics, and/or imaging agents incorporated into the nanoparticles, including but not limited to NO, S—NO, N₂O₃, curcumin, curcumin-selenium complex, antiinflammatories, antimicrobials, antifungals, siRNA, plasmids, nitro fatty acids, imaging probes (e.g., fluorescence, PET).

In accordance with one or more embodiments, described herein are compositions and methods of producing NO-releasing nanoparticles (NO-np). Compositions and methods of producing NO releasing nanoparticles (NO-np) have been described in, for example, PCT International Publication No. PCT/US2015/031907, U.S. Patent Application Publication No. 2015/0147396, and PCT International Application No. PCT/US15/35299, the contents of which are herein incorporated by reference in their entireties.

For example, NO-np can be formed of nitric oxide encapsulated in a matrix of chitosan, polyethylene glycol (PEG) and/or polyvinyl alcohol (PVA), and Tetramethoxysilane (TMOS) or Tetraethoxysilane (TEOS). Another composition for releasing nitric oxide (NO) is formed of nitric oxide encapsulated in a matrix of trehalose, and non-reducing sugar or starch. The composition can further include nitrite, reducing sugar, and/or chitosan. Another composition for releasing nitric oxide (NO) includes nitrite; reducing sugar; chitosan; polyethylene glycol (PEG) and/or polyvinyl alcohol (PVA); Tetramethoxysilane (TMOS) or Tetraethoxysilane (TEOS); and nitric oxide encapsulated in a matrix of chitosan, PEG and TMOS. Another composition for releasing nitric oxide (NO) includes nitrite; reducing sugar; chitosan; trehalose; a non-reducing sugar or starch; and nitric oxide encapsulated in a matrix of trehalose and the non-reducing sugar or starch. Another composition includes nitrite, reducing sugar, chitosan, polyethylene glycol (PEG) and Tetramethoxysilane (TMOS) or Tetraethoxysilane (TEOS), and a composition comprising nitrite, reducing sugar, chitosan, trehalose, and non-reducing sugar or starch. Nitric oxide is released when the composition is exposed to an aqueous environment.

In certain embodiments, the NO-nps can be administered intravenously at a level up to 10 mg/kg of body weight of a subject.

The nanoparticles can be formed of, for example, silica, chitosan, polyethylene glycol, nitrite, glucose, hydrolyzed tetramethoxysilane (TMOS) and hydrolyzed 3-mercaptopropyltrimethoxysilane (MPTS). The nanoparticles can also be formed of, for example, silica, chitosan, polyethylene glycol, nitrite, glucose, hydrolyzed tetramethoxysilane (TMOS) and S-nitroso-N-acetyl cysteine (NAC) and/or S-nitroso-captopril.

NO-np can include a silane in addition to TMOS or TEOS. The additional silane can be chosen, for example, to either alter the internal environment of the resulting particles with respect to properties such as hydrophobicity and polarity or to introduce reactive groups (e.g. amino, carboxyl, sulthydryl) that allow the covalent attachment of additional molecules to the particles. The additional silane can be, for example, a hydrophobic silane, such as, for example, trimethoxyalkyl isopropyl silane, trimethoxyalkyl butyl silane or trimethoxyalkyl fluoropropyl silane.

In at least one embodiment, the NO-np can be a paramagnetic hydrogel hybrid nanoparticle, which can be more uniform with respect to size distribution and more compact with respect to the internal polymeric network, which can result in a slower release profile. This embodiment can also include alcohol to reduce water content and thereby enhance the hydrogen bonding network due to water of the nanoparticles. Toxicity due to the use of alcohol is not an issue because of the lyophilization process, which removes all volatile liquids including free water and alcohol. Further, amine groups can also be incorporated into the polymeric network through the addition of amine-containing silanes (e.g., aminopropyltrimethoxysilane) with TMOS or TEOS, which are used to generate the hydrogel polymeric network. The addition of amine-containing silanes can accelerate the polymerization process, contribute to a tighter internal hydrogen bonding network, and help in PEG conjugation on the surface of the nanoparticles as a means of extending systemic circulation time and increasing the probability of localization at a site with leaky vasculature.

Other modifications to the paramagnetic hydrogel hybrid NO-np can include the introduction of oleic acid or conjugated linoleic acid, and/or other unsaturated fatty acids. When these are included in the NO-np, the resulting nanoparticles contain nitro fatty acids, which are highly anti-inflammatory and potentially chemotherapeutic. Alternatively, nitro fatty acids can be prepared and then incorporated into the recipe for generating the nanoparticles. The introduction of oleic acid or conjugated linoleic acid, and/or other unsaturated fatty acids into the NO-np provides a lipophilic interior to the nanoparticles that will enhance loading of lipophilic deliverables. Further, modified NO-nps with added oleic acid or conjugated linoleic acid, and/or other unsaturated fatty acids can enhance uptake of the NO-np from the gut subsequent to oral intake.

Another modification to the paramagnetic hydrogel hybrid NO-np include doping the TMOS or TEOS with trimethoxy silane derivates that at their fourth conjugation site (e.g., Si(OCH3)3(X)) contains derivatives such as a thiol containing side chain, a lipid containing side chain, a PEG containing side chain, or an alkyl side chain of variable length. Other additives can also be added to the paramagnetic NOnp to enhance the physical properties of the paramagnetic NOnps, such as polyvinyl alcohols.

One method for preparing a paramagnetic hydrogel hybrid NO-np comprises, for example: (a) hydrolyzing Tetramethyl Orthosilicate (TMOS); (b) mixing the sol-gel components; (c) lyophilizing the sol-gel; (d) ball-milling the lyophilized sol-gel particles; and (e) PEGylating of the nanoparticles. Specifically, 5 ml of TMOS, 600 μl of deioinized water, and 560 μl of 2 mM hydrochloric acid are added to a small vial. The contents of the vial are then sonicated approximately 20-30 minutes to get a clear solution and placed on ice. A separate solution of 800 mg of Gadolinium chloride hexahydrate and 200 mg of europium chloride hexahydrate are then solubilized in 6-8 ml of deionized water followed by sequential addition and mixing of 1 ml of PEG-200, 1 ml (1 mg/ml) of either chitosan or water soluble chitosan (depending on the application and usage), and 30 ml of methanol. The resulting mixture is then vortexed thoroughly. Then, 2 ml of the previously hydrolyzed TMOS is added to the solution along with approximately 75-150 μl of 3-aminopropyltrimethoxysilane followed by constant stirring. 4 to 6 ml of ammonium hydroxide is added to the above admixture to form gel followed by vigorous vortexing until complete gelation. The hydroxide creates paramagnetic gadolinium/europium hydroxide that is distributed throughout the resulting hydrogel. The hydroxide also accelerates polymerization which favors small polymers leading to smaller nanoparticles. The resulting gelled material is then lyophilized for 24-48 hours, which removes all volatile component including the methanol. Following lyophilization, the dry material is ball milled at 150 rpm for 8 hours. The resulting material is a very fine white powder. Finally, PEGylation of the paramagnetic nanoparticles is achieved by mixing a suspension of the nanoparticles with an amine binding PEG. Similarly, peptides can be bound to the surface via reaction with the amines on the surface of the nanoparticle. This process can be carried out in water, alcohol or DMSO depending on the nature of the deliverable. Water will initiate release for nitric oxide, and thus the PEGylation needs to be carried out in DMSO which does not result in release of NO. Once the reaction is complete, the PEGylated nanoparticles can be redried and then stored as a dry powder. In an alternative embodiment, thiols can be incorporated into the nanoparticle by using thiol-containing silanes in a manner similar to the process of introducing amnines.

Another method for preparing a paramagnetic hydrogel hybrid NOnp comprises, for example: (a) hydrolyzing TMOS; (b) mixing the sol-gel components; (c) washing the sol-gel; (d) lyophilizing the sol-gel; and (e) ball-milling the sol-gel particles. Specifically, 5 ml of TMOS, 600 μl of deioinized water, and 560 μl of 2 mM hydrochloric acid are added to a small vial. The contents of the vial are then sonicated approximately 20-30 minutes to get a clear solution and placed on ice. 28 ml of methanol, 1 mL of polyvinyl alcohol (PVA) from stock solution (10 mg/mL in deionized water), 2 ml of 300 mM Tris (HCl) buffer at pH 7.5, 1 ml of glycerol, 4 ml of chitosan (1 mg/ml), and 2.76 g of sodium nitrite are then dissolved in the mixture in the above order, and vortexed thoroughly. Then, 4 ml of previously hydrolyzed TMOS is added to the tube, and the contents are vortexed for about two minutes. The tube is allowed to sit undisturbed for gelation. It forms gel in 5 to 10 min. The resulting sol-gel is crushed and deionized water is added until the tube is nearly full. The contents are then vortexed until the mixture is relatively homogeneous. Then, the mixture is centrifuged at 6,000 rpm for 25 minutes, and the supernatant is removed. The gel is then lyophilized for 24-48 hrs. Finally, the resulting particles were ball milled at 150 rpm for 3 hours.

A method for preparing a paramagnetic hydrogel hybrid NO-np with added conjugated linoleic acid comprises, for example: (a) hydrolyzing TMOS; (b) mixing the sol-gel components; (c) lyophilizing the sol-gel; and (d) ball-milling the sol-gel particles. Specifically, 5 ml of TMOS, 600 μl of deioinized water, and 560 μl of 2 mM hydrochloric acid are added to a small vial. The contents of the vial are then sonicated approximately 20-30 minutes to get a clear solution and placed on ice. 1 ml of conjugated linoleic acid (sigma) in DMSO (1:19 v/v ratio in stock), 1.49 g of sodium nitrite (dissolved in 4 ml of PBS buffer at pH 7.5), 1 ml of PEG-200, 8000 μl of chitosan (1 mg/ml), and 28 ml of methanol are then mixed in the above order and vortexed thoroughly. Then, 2 ml of previously hydrolyzed TMOS is added to the solution, and 50-75 μl of 3-aminopropyltrimethoxysilane is added followed by vigorous vortexing until complete gelation. The gel was then lyophilized for 24-48 hrs, and the resulting particles were ball milled at 150 rpm for 8 hours.

One method for preparing NO-np comprises, for example: (a) admixing nitrite, reducing sugar, chitosan, polyethylene glycol (PEG) and/or polyvinyl alcohol (PVA), and Tetramethoxysilane (TMOS) or Tetraethoxysilane (TEOS); (b) drying the mixture of step (a) to produce a gel; and (c) heating the gel until the gel is reduced to a powdery solid. The nitrite is reduced to nitric oxide by the reducing sugar, and nitric oxide is encapsulated in the powdery solid. The encapsulated nitric oxide is released when the composition is exposed to an aqueous environment. The solid of step (c) can be ground to produce particles of a desired size. Preferably, the gel is heated in step (c) to a temperature of 55-70° C., more preferably to about 600° C. Preferably, the gel is heated in step (c) for 24-28 hours. Another method for preparing a composition for releasing nitric oxide (NO) comprises: (a) admixing nitrite, reducing sugar, chitosan, trehalose, and non-reducing sugar or starch; (b) drying the mixture of step (a) to produce a film; and (c) heating the film to form a glassy film. The nitrite is reduced to nitric oxide by the reducing sugar, and nitric oxide is encapsulated in the glassy film. The encapsulated nitric oxide is released when the composition is exposed to an aqueous environment. Preferably, the film is heated in step (c) to a temperature of 55-70° C., more preferably to about 65° C. Preferably, the film is heated in step (c) for about 45 minutes. Preferably, the nitrite is a monovalent or divalent cation salt of nitrite, including for example, one or more of sodium nitrite, calcium nitrite, potassium nitrite, and magnesium nitrite. Preferably, the concentration of nitrite in the composition is 20 nM to about 1 M. The gel can also be lyophilized to produce a particulate material. Alternatively, the mixture may be spray dried to produce a particulate material.

Preferably, the chitosan is at least 50% deacetylated. More preferably, the chitosan is at least 80% deacetylated. Most preferably, the chitosan is at least 85% deacetylated. Preferably, the concentration of chitosan in the composition is 0.05 g-1 g chitosan/100 ml of composition (dry weight). Preferably, the concentration of TMOS or TEOS in the composition is 0.5 ml-5 ml of TMOS or TEOS/24 ml of composition (dry weight).

Preferably, the polyethylene glycol (PEG) has a molecular weight of 200 to 20,000 Daltons, more preferably 200-10,000 Daltons, and most preferably 200-5,000 Daltons. In different embodiments, the PEG can have a molecular weight of, for example, 200-400 Daltons or 3,000-5,000 Daltons. A preferred polyethylene glycol has a molecular weight of 400 Daltons. PEGs of various molecular weights, conjugated to various groups, can be obtained commercially (see, for example, Nektar Therapeutics, Huntsville, Ala.). Preferably, the concentration of polyethylene glycol (PEG) in the composition is 1-5 ml of PEG/24 ml of composition (dry weight).

The nanoparticles can be formed in sizes having a diameter in dry form, for example, of 10 nm to 1,000 μm, preferably 10 nm to 100 μm, or 10 nm to 1 μm, or 10 nm to 500 nm, or 10 nm to 100 nm. Preferably, the nanoparticles have an average diameter of less than about 500 nm, more preferably less than about 250 nm, and most preferably less than about 150 nm. Preferably, NOnp are nontoxic, nonimmunogenic and biodegradable.

Also disclosed herein is a method of preparing nanoparticles comprising S-nitrosothiol (SNO) groups covalently bonded to the nanoparticles, the method comprising: a) providing a buffer solution comprising chitosan, polyethylene glycol, nitrite, glucose, and hydrolyzed 3-mercaptopropyltrimethoxysilane (MPTS); b) adding TMOS to the buffer solution to produce a sol-gel; and c) lyophilizing and ball milling the sol-gel to produce nanoparticles of a desired size.

Provided herein is a method of preparing nanoparticles including a S-nitrosothiol containing molecule encapsulated within the nanoparticle, the method including: a) providing a buffer solution comprising chitosan, polyethylene glycol, nitrite, glucose, and a S-nitrosothiol containing molecule; b) adding TMOS to the buffer solution to produce a sol-gel; and lyophilizing and ball milling the sol-gel to produce nanoparticles of a desired size. The S-nitrosothiol containing molecule encapsulated within the nanoparticle can be, for example, S-nitroso-N-acetyl cysteine (NAC-SNO) and/or S-nitroso-captopril (captopril-SNO).

Preferably MPTS is hydrolyzed with HCl by sonication on an ice-bath. Preferably, TMOS is hydrolyzed with HCL by sonication on an ice-bath.

In accordance with one or more embodiments, the nanoparticles encapsulating S-nitrosothiol-containing molecules are easily produced in small or bulk scale for commercial purposes and are relatively inexpensive. Furthermore, the NO or S-nitrosothiols nanoparticles are stable over a wide range of temperatures, when maintained in a dry and sealed environment. There has been no evidence of any toxicity in extensive animal studies, including in large mammal studies using pigs. The unique aspects of this formulation include: i) long circulation time; ii) the capability for slow sustained release of therapeutically effective levels of nitric oxide within the vasculature; and iii) a platform that is amenable to modifications for fine-tuning of delivery rates and circulation time. Additionally, the platform accommodates the delivery of S-nitrosothiols (e.g., NACSNO) and Captopril-SNO. S-nitrosothiols can be viewed as long lived bioactive forms of nitric oxide. Systemic studies using nanoparticles containing NAC-SNO also support NO-like efficacy in the vasculature. Treatment and administration can proceed by any suitable route, including by introducing the nitric oxide releasing nanoparticles into an IV infusion or transmucosal systemic delivery via sublingual gel or rectal suppository, or combinations of these delivery methods.

Other embodiments include mixing the NO-np with glutathione or other small thiol containing molecules, PEGylating the surface of the NO-np to minimize aggregation, Use of powders derived from nitrite containing trehalose/sugar mixtures that provide for thermal reduction of nitrite to NO. These glassy powders will release NO in a burst mode as they melt when added to an aqueous environment.

Further provided herein is a pharmaceutical composition comprising any of the nanoparticles disclosed herein and a pharmaceutically acceptable carrier.

The nanoparticles described herein can be delivered to a subject by a variety of topical or systemic routes of delivery, including but not limited to percutaneous, inhalation, oral, local injection and intravenous introduction. The nanoparticles can be incorporated, for example, in a cream, ointment, transdermal patch, implantable biomedical device or scrub.

Controlled, sustained release of NO may achieved from a stable, dry powder. This powder may be comprised of nanoparticles for releasing NO. The capacity of these particles to retain and gradually release NO arises from their having combined features of both glassy matrices and hydrogels. This feature allows both for the generation of NO through the thermal reduction of added nitrite by glucose and for the retention of the generated NO within the dry particles. Exposure of these robust biocompatible nanoparticles to moisture initiates the sustained release of the trapped NO over extended time periods as determined both fluorimetrically and amperometrically. The slow sustained release is in contrast to the much faster release pattern associated with the hydration-initialed NO release in powders derived from glassy matrices. These glasses are prepared using trehalose and sucrose doped with either glucose or tagatose as the source of thermal electrons needed to convert nitrite to NO. Significantly, the release profiles for the NO in the hydrogel/glass composite materials are found to be an easily tuned parameter that is modulated through the specific additives used in preparing the hydrogel/glass composites.

In one embodiment, the nanoparticle is stored in a non-aqueous solution. In certain embodiment, the nanoparticle is stored in a buffer suitable for administration in human. In certain embodiment, the nanoparticle is stored in plasma. In certain embodiment, the nanoparticle is stored in the absence of aqueous solution. In certain embodiment, the nanoparticle is stored in the absence of water.

The infusion of NO-nps either intraperitoneally (IP) or intravenously (IV) is also anti-inflammatory, induces vasodilatation, and enhances tissue perfusion by enhancing functional capillary density. Furthermore, NO-np can prevent the inflammatory cascade associated with hemorrhagic shock. In other embodiments, where the nanoparticles release the S-nitrosothiol derivative of N-acetylcysteine (NAC), substantially the same results are achieved.

In certain embodiments, also described herein is a method of making curcumin-loaded nanoparticles. In one or more embodiments, the curcumin-loaded nanoparticles comprise a matrix of chitosan, polyethylene glycol (PEG) and TMOS (or TEOS) encapsulating the curcumin. For example, one method for preparing curcumin-loaded nanoparticles comprises the following. First, tetramethyl orthosilicate (TMOS) is hydrolyzed by adding HCl, followed by 20-minute sonication in ice water bath. The mixture is then refrigerated at 4° C. until monophasic. Curcumin is then dissolved in methanol and combined with chitosan (4.4%), polyethylene glycol (4.4%) and TMOS-HCl (8.8%) to induce polymerization. The gel is then lyophilized at ˜200 mTorr for 48-72 hours, removing all traces of methanol. The resulting powder is processed in a ball mill for ten 30-minute cycles to achieve smaller size and uniform distribution.

In certain embodiments, the curcumin-loaded nanoparticles can also be loaded with selenium. Selenium depletion is a known factor in promoting vascular inflammation. For example, infection with Ebola virus depletes selenium stores thus promoting the inflammatory cascade associated with Ebola infection. As such, the formulation of curcumin-selenium loaded nanoparticles of the present application can ameliorate vascular inflammation.

In certain embodiments, the curcumin-selenium loaded nanoparticles are prepared using the same method as described for the curcumin-loaded nanoparticles above, except that instead of dissolving pure curcumin, a curcumin-selenium complex (selenium tetrachloride to curcumin molar ratio 1:4 up to a total concentration of 0.05 mg of curcumnin-seleniumn complex/mg of nanoparticles) is dissolved. The only additional change to the method of making for the curcumin-selenium loaded nanoparticles is that the step of combining all the components together to induce polymerization (i.e., to form a gel) is done under conditions of continuous sonication, which is maintained until the occurrence of gelation.

In certain embodiments, curcumin-loaded nanoparticles (and curcumin-selenium loaded nanoparticles) can also comprise oleic acid or conjugated linoleic acid, and/or other unsaturated fatty acids to enhance intestinal uptake of nanoparticles for oral delivery.

In certain embodiments, curcumin-loaded nanoparticles (and curcumin-selenium loaded nanoparticles) can be mixed with coconut oil. In this embodiment, the curcumin-loaded nanoparticles are made in accordance with the above procedure, except that the nanoparticles are uniformly mixed into powdered coconut oil and compacted into a suitable block or roll on configuration for topical application. In at least one embodiment, melted coconut oil can be used in place of powder coconut oil. The use of the melted coconut oil in this case is limited because there is some release of curcumin from the nanoparticles once they are mixed into liquid coconut oil. In contrast there is no release when the nanoparticles are mixed with the powdered form of the coconut oil. In certain embodiments, colorless curcumin or chemically modified curcumin can be used. In certain embodiments, other oils or mixtures with other oils (e.g., butter of cacao mixed with coconut oil) can be used to improve the consistency and melting temperature of the solid formulation.

In certain embodiments, free curcumin rather than curcumin-loaded nanoparticles can be used as treatment. In one embodiment, either free curcumin or curcumin-loaded nanoparticles in coconut oil can be delivered to a subject topically or sublingually.

In certain embodiments, curcumin-loaded nanoparticles (and curcumin-selenium loaded nanoparticles) can be delivered to a subject in various ways, including but not limited to intravenously and topically. Other forms of parenterally administered of curcumin and curcumin-loaded nanoparticles can include liposomal delivery vehicles.

Provided herein is a composition comprising a modified nanoparticle (e.g., NO-np, curcumin-loaded nanoparticle, curcumin/selenium loaded nanoparticle) as described herein. The composition further comprises a non-aqueous solution. In certain embodiments, the composition further comprises a buffer suitable for administration in human. In certain embodiments, the composition further comprises plasma. In certain embodiments, the composition further comprises red blood cells. In certain embodiments, the composition does not contain an aqueous solution. In certain embodiments, the composition does not contain water.

In certain embodiments, the composition comprises modified nanoparticles and one or more of the following: Dextrose (in the range of 50-70 mM, 70-90 mM, 90-100 mM, 100-120 mM or 120-150 mM), Adenine (0.1-0.5 mM, 0.5-1 mM, 1-2 mM or 2-3 mM), Monobasic sodium phosphate (0.1-0.5 mM, 0.5-1 mM, 1-5 mM, 5-10 mM, 10-15 mM, 15-20 mM or 20-25 mM), Mannitol (0.1-0.5 mM, 0.5-1 mM, 1-5 mM, 5-10 mM, 10-15 mM, 15-20 mM, 20-25 mM, 25-50 mM, 50-80 mM, 80-100 mM), Sodium citrate (0.5-1 mM, 1-5 mM, 5-10 mM, 10-15 mM, 15-20 mM or 20-25 mM), Citric acid (2.0-2.5 mM), glucose (0.5-1 mM, 1-5 mM, 5-10 mM, 10-15 mM, 15-20 mM or 20-25 mM or 25-40 mM).

In certain embodiments, the concentration of modified nanoparticles in a composition is 0.01-0.02, 0.02-0.05, 0.05-0.08, 0.08-0.1, 0.1-0.12, 0.12-0.15, 0.15-0.18, 0.18-0.2, 0.2-0.23, 0.23-0.25 mg/ml. In certain embodiment, the concentration of nanoparticles in a composition is 0.08-0.12 mg/ml. In certain embodiment, the concentration of nanoparticles in a composition is 0.05-0.1 mg/ml. In one embodiment, the concentration of nanoparticles is 0.8 mg/ml.

In certain embodiments, the modified nanoparticles comprises 10-20, 20-30, 30-40, 40-50, 50-60, 60-70, 70-80, 80-90, 90-100 μg of therapeutic agent (e.g., nitric oxide, curcumin, curcumin/selenium complex) per mg of nanoparticle. In certain embodiments, the modified nanoparticles comprise 22-44, 24-40, 50-60 μg of therapeutic agent per mg of nanoparticle.

In certain embodiments, the modified nanoparticles comprise 10-20, 20-30, 30-40, 40-50, 50-60, 60-70, 70-80, 80-90, 90-100 μg of therapeutic agent per mg of nanoparticle per unit time. In certain embodiments, the modified nanoparticles comprises 22-44, 24-40, 50-60 μg of therapeutic agent per mg of nanoparticle per unit time. In certain embodiment, the unit time is 1-5, 5-10, 10-15, 15-20, 20-25, 25-30, 30-35, 35-40, 40-45, 45-50, 50-60 secs, 1-2 mins, 2-5 mins, 5-10 mins, 10-30 mins, 30-60 mins.

In certain embodiments, the modified nanoparticles have a core size of 50-60, 60-70, 70-80, 80-90, 90-100, 100-110, 110-120, 120-130, 130-140, 140-150, 150-160, 160-170, 170-180, 180-190, 190-200, 200-300, 300-400, and 400-500 nm. In certain embodiment, modified nanoparticles have a core size of 70-150 nm.

In certain embodiments, the modified nanoparticles comprises 2, 3, 4, 5, 6, 7, 8, 9, 10, 20, 30, 40, 50 folds more therapeutic agents than nanoparticles that do not have the modification(s) described in the present disclosure.

In certain embodiments, the modified nanoparticles as disclosed herein have improved permeability crossing the blood brain barrier as compared to other nanoparticles having similar size. In certain embodiments, the modified nanoparticles have a nanoparticle core that has similar size as other previously known nanoparticles and yet has an increased permeability crossing the blood brain barrier by the order of at least 10, 10-10², 10²-10³, 10³-10⁴, 10⁴-10⁵. In certain embodiments, the modified nanoparticles are 2, 3, 4, 5, 6, 7, 8, 9, 10, 20, 30, 40, 50 folds more efficient in penetration across the blood brain barrier than nanoparticles that does not have the modification(s) described in the present disclosure.

In certain embodiments, the modified nanoparticles are 2, 3, 4, 5, 6, 7, 8, 9, 10, 20, 30, 40, 50 folds more efficient in entering a cell at the location that the nanoparticles are targeted in a subject than nanoparticles that do not have the modification(s) described in the present disclosure. In certain embodiments, the cells are cancer cells. In certain embodiments, the cells are glioblastoma cells. In certain embodiments, the cells are cardiac cells, blood vessel cells and capillary cells. In certain embodiments, the cells are bone marrow, spleen, brain, bone, etc.

In certain embodiments, the modified nanoparticles have a size dispersion of 0-5%, 5-15%, 15-20%, 20-25% and 25-30%. In certain embodiments, the modified nanoparticles have a size dispersion of less than 1%. In certain embodiments, the modified nanoparticles have a size dispersion of less than 0.1%.

In certain embodiments, the modified nanoparticles of the present application can be formed in sizes having a diameter in dry form, for example, of 10 nm to 1000 μm, preferably 10 nm to 100 μm, or 10 nm to 1 μm, or 10 nm to 500 nm, or 10 nm to 100 nm. Preferably, the nanoparticles have an average diameter of less than 500 nm.

In certain embodiments, the targeted systemic (vascular) inflammation is highly localized. In certain embodiments, the targeted systemic inflammation is localized to certain cells in the subject. In certain embodiments, the systemic inflammation is localized to 1-10 cells, 10-50 cells, 50-100 cells, 100-500 cells, 500-1,000 cells, 1,000-2,000 cells, 2,000-5,000 cells, 5,000-10,000 cells, 10⁴-10⁵ cells, 10⁵-10⁶ cells, 10⁶-10⁷ cells, 10⁷-10⁸ cells or 10⁹-10¹⁰ cells.

In certain embodiments, the systemic inflammation is localized to 0.1-0.5 mm, 0.5-1 mm, 1-2 mm, 2-3 mm or 3-4 mm.

In certain embodiments, systemic inflammation is detected in a subject by the progressive increase in cytokines over time. In certain embodiments, the cytokines used to detect systemic inflammation are TNFα, TGFβ, MCP-1, IL-1α, IL-1β, IL-4, IL-6, IL-10, and/or IL-12 or any combination thereof. In certain embodiments, the cytokines used to detect systemic inflammation are proinflammatory cytokines (e.g., TNFα, IL-1β and IL-6). In certain embodiments, the cytokines used to detect systemic inflammation are repair cytokines (e.g., IL-4 and IL-10).

In certain embodiments, administration of the modified nanoparticles of the present application can result in the reduction of TNFα, TGFβ, MCP-1, IL-α, IL-1β, IL-4, IL-6, IL-10, and/or IL-1 cytokines in a subject with systemic inflammation. In certain embodiments, one or more of these cytokines are reduced by 1-5%, 5-10%, 10-20%, 20-30%, 30-40%, 40-50%, 50-60%, 60-70%, 70-80%, 80-90%, or 90-95%. In certain embodiments, the one or more of these cytokines are reduced by 0-100 pg/mL, 100-200 pg/mL, 200-300 pg/mL, 300-400 pg/mL, 400-500 pg/mL, 500-600 pg/mL, 600-700 pg/mL, 700-800 pg/mL, 800-900 pg/mL, 900-1000 pg/mL, 1000-1100 pg/mL, 1100-1200 pg/mL, 1200-1300 pg/mL, 1300-1400 pg/mL, 1400-1500 pg/mL, 1500-1600 pg/mL, 1600-1700 pg/mL, 1700-1800 pg/mL, 1800-1900 pg/mL, or 1900-2000 pg/mL.

In certain embodiments, administration of the modified nanoparticles of the present application can result in the reduction of proinflammatory cytokines (e.g., TNFα, IL-1β and IL-6) in a subject with systemic inflammation. In certain embodiments, the proinflammatory cytokines are reduced by 1-5%, 5-10%, 10-20%, 20-30%, 30-40%, 40-50%, 50-60%, 60-70%, 70-80%, 80-90%, or 90-95%. In certain embodiments, the proinflammatory cytokines are reduced by 0-100 pg/mL, 100-200 pg/mL, 200-300 pg/mL, 300-400 pg/mL, 400-500 pg/mL, 500-600 pg/mL, 600-700 pg/mL, 700-800 pg/mL, 800-900 pg/mL, 900-1000 pg/mL, 1000-1100 pg/mL, 1100-1200 pg/mL, 1200-1300 pg/mL, 1300-1400 pg/mL, 1400-1500 pg/mL, 1500-1600 pg/mL, 1600-1700 pg/mL, 1700-1800 pg/mL, 1800-1900 pg/mL, or 1900-2000 pg/mL.

In certain embodiments, systemic inflammation is detected in a subject by an increase in vascular permeability, a decrease in heart rate, an increase in arteriolar diameter, a decrease in arteriolar blood flow, and/or a decrease in functional capillary density.

In certain embodiments, administration of the modified nanoparticles of the present application can result in a decrease in vascular permeability in a subject with systemic inflammation. In certain embodiments, vascular permeability is reduced by 1-5%, 5-10%, 10-15%, 15-20%, 20-25%, 25-30%, 30-35%, 35-40%, 40-45%, or 45-50%. In certain embodiments, vascular permeability is reduced by 1-10%, 10-20%, 20-30%, 30-40%, or 40-50%.

In certain embodiments, administration of the modified nanoparticles of the present application can result in an increase in heart rate in a subject with systemic inflammation. In certain embodiments, heart rate is increased by 1-5%, 5-10%, 10-15%, 15-20%, 20-25%, 25-30%, 30-35%, 35-40%, 40-45%, or 45-50%. In certain embodiments, heart rate is increased by 1-10%, 10-20%, 20-30%, 30-40%, or 40-50%. In certain embodiments, heart rate is increased by 1-5 bpm, 5-10 bpm, 10-15 bpm, 15-20 bpm, 20-25 bpm, 25-30 bpm, 30-35 bpm, 35-40 bpm, 40-45 bpm, 45-50 bpm, 50-55 bpm, 55-60 bpm, 60-65 bpm, 65-70 bpm, 70-75 bpm, 75-80 bpm, 80-85 bpm, 85-90 bpm, 90-95 bpm, or 95-100 bpm.

In certain embodiments, administration of the modified nanoparticles of the present application can result in a decrease in arteriolar diameter in a subject with systemic inflammation. In certain embodiments, arteriolar diameter is reduced by 1-2%, 2-4%, 4-6%, 6-8%, 8-100%, 10-12%, 12-14%, 14-16%, 16-18%, 18-20%, 20-22%, 22-24%, 24-26%, 26-28%, or 28-30%. In certain embodiments, arteriolar diameter is reduced by 1-5%, 5-10%, 10-15%, 15-20%, 20-25%, 25-30%, 30-35%, 35-40%, 40-45%, or 45-50%.

In certain embodiments, administration of the modified nanoparticles of the present application can result in an increase in arteriolar blood flow in a subject with systemic inflammation. In certain embodiments, arteriolar blood flow is increased 1-5%, 5-10%, 10-15%, 15-20%, 20-25%, 25-30%, 30-35%, 35-40%, 40-45%, 45-50%, 50-55%, 55-60%, 60-65%, or 65-70%. In certain embodiments, arteriolar blood flow is increased 1-10%, 10-20%, 20-30%, 30-40%, 40-50%, 50-60%, or 60-70%.

In certain embodiments, administration of the modified nanoparticles of the present application can result in an increase in functional capillary density in a subject with systemic inflammation. In certain embodiments, functional capillary density is increased 1-5%, 5-10%, 10-20%, 20-30%, 30-40%, 40-50%, 50-60%, 60-70%, 70-80%, 80-90%, 90-100%, 100-110%, 110-120%, 120-130%, 130-140%, 140-150%, 150-160%, 160-170%, 170-180%, 180-190%, or 190-200%. In certain embodiments, functional capillary density is increased 50-100%, 100-150%, or 150-200%. In certain embodiments, functional capillary density is increased 100-200%.

In certain embodiments, the modified nanoparticles of the present application are administered prior to the onset of systemic inflammation. In certain embodiments, the modified nanoparticles of the present application are administered prior to infection with a Filovirus (e.g., Ebola virus, Marburg virus). In certain embodiments, the modified nanoparticles of the present application are administered after onset of systemic inflammation. In certain embodiments, the modified nanoparticles of the present application are administered within 48 hours, 24 hours, or 12 hours of the onset of systemic inflammation. In certain embodiments, the modified nanoparticles of the present application are administered 1-12 hours, 12-24 hours, 24-36 hours, or 36-48 hours after onset of systemic inflammation. In certain embodiments, the modified nanoparticles of the present application are administered 1-2 hours, 2-3 hours, 3-4 hours, 4-5 hours, 5-6 hours, 6-7 hours, 7-8 hours, 8-9 hours, 9-10 hours, 10-11 hours, 11-12 hours, 12-13 hours, 13-14 hours, 14-15 hours, 15-16 hours, 16-17 hours, 17-18 hours, 18-19 hours, 19-20 hours, 20-21 hours, 21-22 hours, 22-23 hours, or 23-24 hours after onset of systemic inflammation. In certain embodiments, the modified nanoparticles of the present application are administered prior to the increase of cytokines associated with systemic inflammation.

In another aspect, the present invention provides any of the nanoparticle compositions described herein in kits, optionally including instructions for use of the nanoparticle compositions. That is, the kit can include a description of use of a composition in any method described herein. A “kit,” as used herein, typically defines a package, assembly, or container (such as an insulated container) including one or more of the components of the invention, and/or other components associated with the invention, for example, as previously described. Each of the components of the kit may be provided in liquid form (e.g., in solution), or in solid form (e.g., a dried powder, frozen, etc.).

In some cases, the kit includes one or more components, which may be within the same or in two or more receptacles, and/or in any combination thereof. The receptacle is able to contain a liquid, and non-limiting examples include bottles, vials, jars, tubes, flasks, beakers, or the like. In some cases, the receptacle is spill-proof (when closed, liquid cannot exit the receptacle, regardless of orientation of the receptacle).

The NO or S-nitrosothiols nanoparticles of the present application are easily produced in small or bulk scale for commercial purposes and are relatively inexpensive. Furthermore, the NO or S-nitrosothiols nanoparticles are expected to be stable over a wide range of temperatures, when maintained in a dry and sealed environment. Scale up for compassionate care testing is anticipated to be relatively inexpensive and achieved over a very short time period. The dosing based on rodent and pig models can be extrapolated to humans. Treatment and administration will proceed by any suitable route, including by introducing the nitric oxide releasing nanoparticles into an IV infusion or transmucosal systemic delivery via sublingual gel or rectal suppository, or combinations of these delivery methods.

### 5.1 Method of Delivering the Paramagnetic Hybrid Hydrogel Nanoparticles

In accordance with one or more embodiments, provided herein is a method of delivering paramagnetic hybrid hydrogel nanoparticles to a target location in a subject by applying a magnetic field to the subject. Methods of delivering paramagnetic nanoparticles to a target location have been described in, for example, PCT International Application No. PCT/US2015/058605, the contents of which are herein incorporated by reference in its entirety.

For instance, provided herein is a method of delivering compositions, therapeutics, and/or imaging agents including (but not limited to): NO, S—NO, N₂O₃, curcumin, curcumin-selenium complex, antiinflammatories, antimicrobials, antifungals, siRNA, plasmids, nitro fatty acids, imaging probes (e.g., fluorescence, PET) to a predetermined location in a subject comprising administering to the subject a composition as described herein, or paramagnetic nanoparticles comprising the compositions described herein, and applying a magnetic field to the subject, such that the magnetic field is present in the predetermined location at a strength sufficient to attract an administered paramagnetic nanoparticle composition.

In an embodiment, the applied magnetic field thereby delivers the paramagnetic nanoparticles comprising the compositions (or therapeutics or imaging agents) described herein to the predetermined location.

In an embodiment, the paramagnetic nanoparticle composition is administered systemically. In an embodiment, the paramagnetic nanoparticle composition is administered intravenously, by direct injection or catheterization into the predetermined location or in the vicinity thereof. In an embodiment, the magnetic field is applied from one or more magnetic field external to the body of the subject. In an embodiment, the location of the paramagnetic nanoparticles is monitored using MRI. In an embodiment, the paramagnetic nanoparticles comprise fluorophores.

In certain embodiments, the hybrid hydrogel paramagnetic nanoparticles and the methods of delivering the hybrid hydrogel paramagnetic nanoparticles to a target location in a subject can provide an unexpected therapeutic benefit in the treatment of vascular inflammation. In particular, when treating vascular inflammation, treatment with therapeutic compositions (e.g., nanoparticles) should be very targeted to the target location. However, depending on the severity of vascular inflammation, it can be difficult to deliver the therapeutic compositions to the target area, as administered therapeutic compositions have a tendency to migration to other areas of the body due to vascular leakiness. As such, administration of paramagnetic nanoparticles and the application of a magnetic field at the target location provides targeted delivery of therapeutic compositions to the area of vascular inflammation.

### 5.2 Types of Disease and Disorders

The present disclosure provides methods of treating or preventing or managing a disease or disorder in humans by administering to humans in need of such treatment or prevention a pharmaceutical composition comprising an amount of modified nanoparticles effective to treat or prevent the disease or disorder. In other enlbodiments, the disease or disorder is an inflammatory disease or disorder.

The present application encompasses methods for preventing, treating, managing, and/or ameliorating an inflammatory disorder or one or more symptoms thereof as an alternative to other conventional therapies. In specific embodiments, the patient being managed or treated in accordance with the methods of the present application is refractory to other therapies or is susceptible to adverse reactions from such therapies. The patient may be a person with a suppressed immune system (e.g., post-operative patients, chemotherapy patients, and patients with immunodeficiency disease, patients with broncho-pulmonary dysplasia, patients with congenital heart disease, patients with cystic fibrosis, patients with acquired or congenital heart disease, and patients suffering from an infection), a person with impaired renal or liver function, the elderly, children, infants, infants born prematurely, persons with neuropsychiatric disorders or those who take psychotropic drugs, persons with histories of seizures, or persons on medication that would negatively interact with conventional agents used to prevent, manage, treat, or ameliorate a viral respiratory infection or one or more symptoms thereof.

In certain embodiments, the present application provides a method of preventing, treating, managing, and/or ameliorating an autoimmune disorder or one or more symptoms thereof, said method comprising administering to a subject in need thereof a dose of an effective amount of one or more pharmaceutical compositions of the present application. In autoimmune disorders, the immune system triggers an immune response and the body's normally protective immune system causes damage to its own tissues by mistakenly attacking self. There are many different autoimmune disorders which affect the body in different ways. For example, the brain is affected in individuals with multiple sclerosis, the gut is affected in individuals with Crohn's disease, and the synovium, bone and cartilage of various joints are affected in individuals with rheumatoid arthritis. As autoimmune disorders progress, destruction of one or more types of body tissues, abnormal growth of an organ, or changes in organ function may result. The autoimmune disorder may affect only one organ or tissue type or may affect multiple organs and tissues. Organs and tissues commonly affected by autoimmune disorders include red blood cells, blood vessels, connective tissues, endocrine glands (e.g., the thyroid or pancreas), muscles, joints, and skin.

Examples of autoimmune disorders that can be prevented, treated, managed, and/or ameliorated by the methods of the present application include, but are not limited to, adrenergic drug resistance, alopecia areata, ankylosing spondylitis, antiphospholipid syndrome, autoimmune Addison's disease, autoimmune diseases of the adrenal gland, allergic encephalomyelitis, autoimmune hemolytic anemia, autoimmune hepatitis, autoimmune inflammatory eye disease, autoimmune neonatal thrombocytopenia, autoimmune neutropenia, autoimmune oophoritis and orchitis, autoimmune thrombocytopenia, autoimmune thyroiditis, Behcet's disease, bullous pemphigoid, cardiomyopathy, cardiotomy syndrome, celiac sprue-dermatitis, chronic active hepatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, dense deposit disease, discoid lupus, essential mixed cryoglobulinemia, fibromyalgia-fibromyositis, glomerulonephritis (e.g., IgA nephrophathy), gluten-sensitive enteropathy, Goodpasture's syndrome, Graves' disease, Guillain-Barre, hyperthyroidism (i.e., Hashimoto's thyroiditis), idiopathic pulmonary fibrosis, idiopathic Addison's disease, idiopathic thrombocytopenia purpura (ITP), IgA neuropathy, juvenile arthritis, lichen planus, lupus erythematosus, Meniere's disease, mixed connective tissue disease, multiple sclerosis, Myasthenia Gravis, myocarditis, type I or immune-mediated diabetes mellitus, neuritis, other endocrine gland failure, pemphigus vulgaris, pernicious anemia, polyarteritis nodosa, polychrondritis, Polyendocrinopathies, polyglandular syndromes, polymyalgia rheumatica, polymyositis and dermatomyositis, post-MI, primary agammaglobulinemia, primary biliary cirrhosis, psoriasis, psoriatic arthritis, Raynauld's phenomenon, relapsing polychondritis, Reiter's syndrome, rheumatic heart disease, rheumatoid arthritis, sarcoidosis, scleroderma, Sjogren's syndrome, stiff-man syndrome, systemic lupus erythematosus, takayasu arteritis, temporal arteritis/giant cell arteritis, ulcerative colitis, urticaria, uveitis, Uveitis Opthalmia, vasculitides such as dermatitis herpetiformis vasculitis, vitiligo, and Wegener's granulomatosis.

### 5.2.1 Cytokine Response to Systemic Inflammation

Many disease conditions and/or infections involve an inflammatory phase that results in extensive “leakiness” in the vasculature of the infected individual leading to extensive leakage of bodily fluids out of the blood vessels. This kind of leakage is typically caused by an imbalance in the concentration gradient of nitric oxide (NO) surrounding the lining of the blood vessels. Blood vessels are coated on their external side with perivascular cells that migrate in response to the nitric oxide concentration gradients. Under normal conditions, the inner wall (endothelium) of the blood vessels produces nitric oxide whereas there is minimal nitric oxide on the external side resulting. This pattern favors having the perivascular cells forming a tight coating around the blood vessel which prevents leakage. Under inflammatory conditions, the endothelium no longer generates nitric oxide whereas there is an over production of nitric oxide on the external site resulting in the movement of the perivascular cells away from the surface of the blood vessels. Loss of the perivascular coating results in leaky blood vessels.

During inflammation the innate immune system responds by using pattern Toll-like receptors (TLRs) to pathogen-associated molecular patterns. Surface molecules of gram-positive and gram-negative bacteria (peptidoglycan and lipopolysaccharide) bind to TLR-2 and TLR-4, respectively. TLR-2 and TLR-4 binding initiates an intracellular signaling cascade that culminates in nuclear transport of the transcription factor nuclear factor kappa B (NFκB), which triggers transcription of cytokines such as TNFα and interleukin 6 (IL-6). Cytokines up-regulate adhesion molecules of neutrophils and endothelial cells, and neutrophil activation, leading to bacterial clearance.

### 5.2.2 Conditions Characterized by Leaky Vessels or Vasculature

The prevention of fluid loss and tissue swelling is important for human health. The properties of blood vessels are adversely affected in many disease states, including: non-infectious conditions such as: cardiovascular disease, neurological conditions (including various brain cancers such as glioblastoma, as well as neurodegenerative diseases such as Alzheimer's), diabetes, edema, and cancers (especially metastases). In cancer, leaky vessels can lead to metastasis, as well as to leakage of fluid into the lungs. Diabetes is marked by several problems posed by blood vessel leakiness, which can lead to amputation and vision loss, among other complications.

The use of curcumin-loaded nanoparticles, curcumin-selenium loaded nanoparticles, NO-releasing nanoparticles, sustained release NO nanoparticles or S-nitrosothiols (NACSNO), alone or in combination, or with other treatments, may serve to reduce fluid loss and tissue swelling in any of these diseases characterized by “leaky vasculature.”

Many types of infections can lead to edema, shock, or sepsis. Causative agents include gram positive bacteria, gram negative bacteria, anaerobic bacteria, fungal infections, and atypical bacteria (See, for example a description of typical infectious bacteria, viruses, fungal infections and agents used to treat them in U.S. Pat. No. 8,741,942).

A common and often fatal complication of sepsis is acute respiratory distress syndrome (ARDS). In ARDS the vessels in the lungs of sepsis patients become porous, allowing fluid to leak into the lungs and leading to pulmonary edema. As a result, many patients suffering from sepsis have to go on ventilators. Additionally, patients typically receive antibiotics to treat the bacterial or viral infection at the root of the ARDS; however, antibiotics do nothing to alleviate the pulmonary edema and other vasculature complications that are underway in the patient. Thus, the use of sustained release NO nanoparticles, alone or in combination with other treatments, may serve to reduce fluid loss and tissue swelling in any of these diseases characterized by “leaky vasculature.”

Filoviruses (e.g., Ebola virus (EBOV) and Marburg virus (MARV)) are among the most lethal and destructive viruses. They cause severe, often fatal viral hemorrhagic fevers in humans and nonhuman primates (e.g., monkeys, gorillas, and chimpanzees). The incubation period for Filovirus infection ranges from 2 to 21 days. The onset of illness is abrupt and is characterized by high fever, headaches, joint and muscle aches, sore throat, fatigue, diarrhea, vomiting, and stomach pain. A rash, red eyes, hiccups and internal and external bleeding may be seen in some patients. Within one week of becoming infected with the virus, most patients experience chest pains and multiple organ failure, go into shock, and die. Some patients also experience blindness and extensive bleeding before dying.

### 5.3 Mode of Administration

The compositions of the invention can be formulated for administration in any convenient way for use in human or veterinary medicine. The invention therefore includes within its scope pharmaceutical compositions comprising a product of the present invention that is adapted for use in human or veterinary medicine.

In one embodiment, a composition of the present application is administered by introducing a nanoparticle comprising the composition into an IV infusion or transmucosal systemic delivery via sublingual gel or rectal suppository. More specifically, the present compositions, which comprise one or more modified nanoparticles, can be administered by infusion or bolus injection, by absorption through epithelial or mucocutaneous linings (e.g., oral mucosa, rectal and intestinal mucosa, etc.) or orally and may be administered together with another biologically active agent. Administration can be systemic or local. Various delivery systems are known. In certain embodiments, more than one modified nanoparticle is administered to a patient. Methods of administration include but are not limited to intradermal, intramuscular, intraperitoneal, intravenous, subcutaneous, intranasal, epidural, oral, sublingual, intranasal, intra-arteriole, intracerebral, intravaginal, transdermal, rectally, by inhalation, or topically, particularly to the ears, nose, eyes, or skin. If desired, inactivated therapeutic formulations may be injected, e.g., intravascular, intratumor, subcutaneous, intraperitoneal, intramuscular, etc. The preferred mode of administration is left to the discretion of the practitioner, and will depend in-part upon the site of the medical condition. In most instances, administration will result in the release of the modified nanoparticle into the bloodstream.

In specific embodiments, it may be desirable to administer one or more compounds of the present application locally to the area in need of treatment. This may be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site).

Pulmonary administration can also be employed, e.g., by use of an inhaler or nebulizer, and formulation with an aerosolizing agent, or via perfusion in a fluorocarbon or synthetic pulmonary surfactant. In certain embodiments, the compounds of the present application can be formulated as a suppository, with traditional binders and vehicles such as triglycerides.

In yet another embodiment, the compounds of the present application can be delivered in a controlled release system. In one embodiment, a pump may be used (see Langer, supra; Sefton, 1987, CRC Crit. Ref. Biomed. Eng. 14:201; Buchwald et al., 1980, Surgery 88:507 Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled-release system can be placed in proximity of the target of the modified nanoparticle, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

In one embodiment, the pharmaceutical composition is conveniently administered as an oral formulation. Oral dosage forms are well known in the art and include tablets, caplets, gelcaps, capsules, and medical foods. Tablets, for example, can be made by well-known compression techniques using wet, dry, or fluidized bed granulation methods.

Such oral formulations may be presented for use in a conventional manner with the aid of one or more suitable excipients, diluents, and carriers. Pharmaceutically acceptable excipients assist or make possible the formation of a dosage form for a bioactive material and include diluents, binding agents, lubricants, glidants, disintegrants, coloring agents, and other ingredients. Preservatives, stabilizers, dyes and even flavoring agents may be provided in the pharmaceutical composition. Examples of preservatives include sodium benzoate, ascorbic acid and esters of p-hydroxybenzoic acid. Antioxidants and suspending agents may be also used. An excipient is pharmaceutically acceptable if, in addition to performing its desired function, it is non-toxic, well tolerated upon ingestion, and does not interfere with absorption of bioactive materials.

Acceptable excipients, diluents, and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient, diluent, and carrier can be selected with regard to the intended route of administration and standard pharmaceutical practice.

### 5.4 Dosage

The dosage of a therapeutic formulation of the present application will vary widely, depending upon the nature of the disease, the patient's medical history, the frequency of administration, the manner of administration, the clearance of the agent from the host, and the like. The initial dose may be larger, followed by smaller maintenance doses. The dose may be administered as infrequently as weekly or biweekly, or fractionated into smaller doses and administered daily, semi-weekly, etc., to maintain an effective dosage level. In some cases, oral administration will require a higher dose than if administered intravenously. In some cases, topical administration will include application several times a day, as needed, for a number of days or weeks in order to provide an effective topical dose.

More specifically, the amount of a modified nanoparticle that will be effective in the treatment of a particular disorder or condition disclosed herein will depend on the nature of the disorder or condition, and can be determined by standard clinical techniques. In addition, in vitro or in vivo assays may optionally be employed to help identify optimal dosage ranges. The precise dose to be employed in the compositions will also depend on the route of administration, and the seriousness of the disease or disorder, and should be decided according to the judgment of the practitioner and each patient's circumstances. However, suitable dosage ranges for oral administration are generally about 0.001 milligram to 200 milligrams of a compound of the present application per kilogram body weight. In specific preferred embodiments of the present application, the oral dose is 0.01 milligram to 70 milligrams per kilogram body weight, more preferably 0.1 milligram to 50 milligrams per kilogram body weight, more preferably 0.5 milligram to 20 milligrams per kilogram body weight, and yet more preferably 1 milligram to 10 milligrams per kilogram body weight. In another embodiment, the oral dose is 5 milligrams of modified nanoparticle per kilogram body weight. The dosage amounts described herein refer to total amounts administered; that is, if more than one modified nanoparticle is administered, the preferred dosages correspond to the total amount of the modified nanoparticles administered. Oral compositions preferably contain 10% to 95% active ingredient by weight.

Suitable dosage ranges for intravenous (i.v.) administration are 0.01 milligram to 100 milligrams per kilogram body weight, 0.1 milligram to 35 milligrams per kilogram body weight, and 1 milligram to 10 milligrams per kilogram body weight. Suitable dosage ranges for intranasal administration are generally about 0.01 pg/kg body weight to 1 mg/kg body weight. Suppositories generally contain 0.01 milligram to 50 milligrams of modified nanoparticles per kilogram body weight and comprise active ingredient in the range of 0.5% to 10% by weight. Recommended dosages for intradermal, intramuscular, intraperitoneal, subcutaneous, epidural, sublingual, intracerebral, intravaginal, transdermal administration or administration by inhalation are in the range of 0.001 milligram to 200 milligrams per kilogram of body weight. Suitable doses of the modified nanoparticles for topical administration are in the range of 0.001 milligram to 1 milligram, depending on the area to which the compound is administered. Effective doses may be extrapolated from dose-response curves derived from in vitro or animal model test systems. Such animal models and systems are well known in the art.

The present application also provides pharmaceutical packs or kits comprising one or more containers filled with one or more modified nanoparticles. Optionally associated with such container(s) can be a notice in the form prescribed by a governmental agency regulating the manufacture, use or sale of pharmaceuticals or biological products, which notice reflects approval by the agency of manufacture, use or sale for human administration. In a certain embodiment, the kit contains more than one modified nanoparticles. In another embodiment, the kit comprises a modified nanoparticles and a second therapeutic agent.

The modified nanoparticles are preferably assayed in vitro and in vivo, for the desired therapeutic or prophylactic activity, prior to use in humans. For example, in vitro assays can be used to determine whether administration of a specific modified nanoparticle or a combination of modified nanoparticles is preferred for lowering fatty acid synthesis. The modified nanoparticles may also be demonstrated to be effective and safe using animal model systems.

Other methods will be known to the skilled artisan and are within the scope of the present application.

### 5.5 Combination Therapy

In certain embodiments, the modified nanoparticles of the present application can be used in combination therapy with at least one other therapeutic agent. The modified nanoparticles and the therapeutic agent can act additively or, more preferably, synergistically. In a preferred embodiment, a composition comprising a modified nanoparticle is administered concurrently with the administration of another therapeutic agent, which can be part of the same composition as the modified nanoparticle or a different composition. In another embodiment, a composition comprising a modified nanoparticle is administered prior or subsequent to administration of another therapeutic agent. As many of the disorders for which the modified nanoparticles are useful in treating are chronic disorders, in one embodiment combination therapy involves alternating between administering a composition comprising a modified nanoparticle and a composition comprising another therapeutic agent, e.g., to minimize the toxicity associated with a particular drug. The duration of administration of each drug or therapeutic agent can be, e.g., one month, three months, six months, or a year. In certain embodiments, when a modified nanoparticle is administered concurrently with another therapeutic agent that potentially produces adverse side effects including but not limited to toxicity, the therapeutic agent can advantageously be administered at a dose that falls below the threshold at which the adverse side is elicited.

In certain embodiments, the modified nanoparticles of the present application can be administered together with one or more antifungal agents in the form of antifungal cocktails, or individually, but close enough in time to have a synergistic effect on the treatment of the infection. An antifungal cocktail is a mixture of any one of the compounds described herein with another antifungal drug. In one embodiment, a common administration vehicle (e.g., tablet, implants, injectable solution, injectable liposome solution, etc.) is used in for the compound as described herein and other antifungal agent(s).

Anti-fungal agents are useful for the treatment and prevention of infective fungi. Anti-fungal agents can be classified by their mechanism of action. Some anti-fungal agents function as cell wall inhibitors by inhibiting glucose synthase. These include, but are not limited to, basiungin/ECB. Other anti-fungal agents function by destabilizing membrane integrity. These include, but are not limited to, immidazoles, such as clotrimazole, sertaconzole, fluconazole, itraconazole, ketoconazole, miconazole, and voriconacole, as well as FK 463, amphotericin B, BAY 38-9502, MK 991, pradimicin, UK 292, butenafine, and terbinafine. Other anti-fungal agents function by breaking down chitin (e.g. chitinase) or immunosuppression (501 cream).

Other antifungal agents include Acrisorcin; Ambruticin; Amphotericin B; Azaconazole; Azaserine; Basifungin; Bifonazole; Biphenamine Hydrochloride; Bispyrithione Magsulfex; Butoconazole Nitrate; Calcium Undecylenate; Cancidas (Caspofungin Acetate). Candicidin; Carbol-Fuchsin; Chlordantoin; Ciclopirox; Ciclopirox Olamine; Cilofungin; Cisconazole; Clotrimazole; Cuprimyxin; Denofungin; Dipyrithione; Doconazole; Econazole; Econazole Nitrate; Enilconazole; Ethonam Nitrate; Fenticonazole Nitrate; Filipin; Fluconazole; Flucytosine; Fungimycin; Griseofulvin; Hamycin; Isoconazole; Itraconazole; Kalafungin; Ketoconazole; Lomofungin; Lydimycin; Mepartricin; Miconazole; Miconazole Nitrate; Monensin; Monensin Sodium; Naftifine Hydrochloride; Neomycin Undecylenate; Nifuratel; Nifurmerone; Nitralamine Hydrochloride; Nystatin; Octanoic Acid; Orconazole Nitrate; Oxiconazole Nitrate; Oxifungin Hydrochloride; Parconazole Hydrochloride; Partricin; Potassium Iodide; Proclonol; Pyrithione Zinc; Pyrrolnitrin; Rutamycin; Sanguinarium Chloride; Saperconazole; Scopafungin; Selenium Sulfide; Sinefungin; Sulconazole Nitrate; Terbinafine; Terconazole; Thiram; Ticlatone; Tioconazole; Tolciclate; Tolindate; Tolnaftate; Triacetin; Triafungin; Undecylenic Acid; Viridofulvin; Zinc Undecylenate; and Zinoconazole Hydrochloride.

In certain embodiments, the modified nanoparticles described herein can be used in combination with one or more antifungal compounds. These antifungal compounds include but are not limited to: polyenes (e.g., amphotericin b, candicidin, mepartricin, natamycin, and nystatin), allylamines (e.g., butenafine, and naftifine), imidazoles (e.g., bifonazole, butoconazole, chlordantoin, flutrimazole, isoconazole, ketoconazole, and lanoconazole), thiocarbamates (e.g., tolciclate, tolindate, and tolnaftate), triazoles (e.g., fluconazole, itraconazole, saperconazole, and terconazole), bromosalicylchloranilide, buclosamide, calcium propionate, chlorphenesin, ciclopirox, azaserine, griseofulvin, oligomycins, neomycin undecylenate, pyrrolnitrin, siccanin, tubercidin, and viridin. Additional examples of antifungal compounds include but are not limited to Acrisorcin; Ambruticin; Amphotericin B; Azaconazole; Azaserine; Basifungin; Bifonazole; Biphenamine Hydrochloride; Bispyrithione Magsulfex; Butoconazole Nitrate; Calcium Undecylenate; Candicidin; Carbol-Fuchsin; Chlordantoin; Ciclopirox; Ciclopirox Olamine; Cilofungin; Cisconazole; Clotrimazole; Cuprimyxin; Denofungin; Dipyrithione; Doconazole; Econazole; Econazole Nitrate; Enilconazole; Ethonam Nitrate; Fenticonazole Nitrate; Filipin; Fluconazole; Flucytosine; Fungimycin; Griseofulvin; Hamycin; Isoconazole; Itraconazole; Kalafungin; Ketoconazole; Lomofingin; Lydimycin; Mepartricin; Miconazole; Miconazole Nitrate; Monensin; Monensin Sodium; Naftifine Hydrochloride; Neomycin Undecylenate; Nifuratel; Nifurmerone; Nitralamine Hydrochloride; Nystatin; Octanoic Acid; Orconazole Nitrate; Oxiconazole Nitrate; Oxifungin Hydrochloride; Parconazole Hydrochloride; Partricin; Potassium Iodide; Proclonol; Pyrithione Zinc; Pyrrolnitrin; Rutamycin; Sanguinarium Chloride; Saperconazole; Scopafungin; Selenium Sulfide; Sinefungin; Sulconazole Nitrate; Terbinafine; Terconazole; Thiram; Ticlatone; Tioconazole; Tolciclate; Tolindate; Tolnaftate; Triacetin; Triafuigin; Undecylenic Acid; Viridoflilvin; Zinc Undecylenate; and Zinoconazole Hydrochlorid

In certain embodiments, the modified nanoparticles of the present application can be administered together with treatment with irradiation or one or more chemotherapeutic agents. For irridiation treatment, the irradiation can be gamma rays or X-rays. For a general overview of radiation therapy, see Hellman, Chapter 12: Principles of Radiation Therapy Cancer, in: Principles and Practice of Oncology, DeVita et al., eds. 2.nd. Ed., J.B. Lippencott Company, Philadelphia. Useful chemotherapeutic agents include methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, and docetaxel. In a specific embodiment, a composition comprising the modified nanoparticle further comprises one or more chemotherapeutic agents and/or is administered concurrently with radiation therapy. In another specific embodiment, chemotherapy or radiation therapy is administered prior or subsequent to administration of a present composition, preferably at least an hour, five hours, 12 hours, a day, a week, a month, more preferably several months (e.g., up to three months), subsequent to administration of a composition comprising the modified nanoparticle.

Any therapy (e.g., therapeutic or prophylactic agent) which is useful, has been used, or is currently being used for the prevention, treatment, and/or management of a disorder, e.g., cancer, can be used in compositions and methods of the present application. Therapies (e.g., therapeutic or prophylactic agents) include, but are not limited to, peptides, polypeptides, conjugates, nucleic acid molecules, small molecules, mimetic agents, synthetic drugs, inorganic molecules, and organic molecules. Non-limiting examples of cancer therapies include chemotherapies, radiation therapies, hormonal therapies, and/or biological therapies/immunotherapies and surgery. In certain embodiments, a prophylactically and/or therapeutically effective regimen of the present application comprises the administration of a combination of therapies.

Examples of cancer therapies include, but not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bisphosphonates (e.g., pamidronate (Aredria), sodium clondronate (Bonefos), zoledronic acid (Zometa), alendronate (Fosamax), etidronate, ibandornate, cimadronate, risedromate, and tiludromate); bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; EphA2 inhibitors; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alpha-2a; interferon alpha-2b; interferon alpha-n1; interferon alpha-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; anti-CD2 antibodies; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin: plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other examples of cancer therapies include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; Bcl-2 inhibitors; Bcl-2 family inhibitors, including ABT-737; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspennine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cyzarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; HMG CoA reductase inhibitors (e.g., atorvastatin, cerivastatin, fluvastatin, lescol, lupitor, lovastatin, rosuvastatin, and simvastatin); hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; LFA-3TIP; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; nmitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone BI; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; 5-fluorouracil; leucovorin; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; thalidomide; velaresol; veramine; verdins; verteporfin; vinorelbine; vinxaltine; vorozole; zanoterone; zeniplatin; zilascorb; and zinostatin stimalamer.

In some embodiments, the therapy(ies) used in combination with the modified nanoparticles is an immunomodulatory agent. Non-limiting examples of immunomodulatory agents include proteinaceous agents such as cytokines, peptide mimetics, and antibodies (e.g., human, humanized, chimeric, monoclonal, polyclonal, Fvs, ScFvs, Fab or F(ab)2 fragments or epitope binding fragments), nucleic acid molecules (e.g., antisense nucleic acid molecules and triple helices), small molecules, organic compounds, and inorganic compounds. In particular, immunomodulatory agents include, but are not limited to, methotrexate, leflunomide, cyclophosphamide, cytoxan, Immuran, cyclosporine A, minocycline, azathioprine, antibiotics (e.g., FK506 (tacrolimus)), methylprednisolone (MP), corticosteroids, steroids, mycophenolate mofetil, rapamycin (sirolimus), mizoribine, deoxyspergualin, brequinar, malononitriloamindes (e.g., leflunamide). Other examples of immunomodulatory agents can be found, e.g., in U.S. Publ'n No. 2005/0002934 A1 at paragraphs 259-275 which is incorporated herein by reference in its entirety. In one embodiment, the immunomodulatory agent is a chemotherapeutic agent. In an alternative embodiment, the immunomodulatory agent is an immunomodulatory agent other than a chemotherapeutic agent. In some embodiments, the therapy(ies) used in accordance with the present application is not an immunomodulatory agent.

In some embodiments, the therapy(ies) used in combination with the modified nanoparticles is an anti-angiogenic agent. Non-limiting examples of anti-angiogenic agents include proteins, polypeptides, peptides, conjugates, antibodies (e.g., human, humanized, chimeric, monoclonal, polyclonal, Fvs, ScFvs, Fab fragments, F(ab)2 fragments, and antigen-binding fragments thereof) such as antibodies that bind to TNF-alpha, nucleic acid molecules (e.g., antisense molecules or triple helices), organic molecules, inorganic molecules, and small molecules that reduce or inhibit angiogenesis. Other examples of anti-angiogenic agents can be found, e.g., in U.S. Publ'n No. 2005/0002934 A1 at paragraphs 277-282, which is incorporated by reference in its entirety. In other embodiments, the therapy(ies) used in accordance with the present application is not an anti-angiogenic agent.

In some embodiments, the therapy(ies) used in combination with the modified nanoparticles is an anti-inflammatory agent. Non-limiting examples of anti-inflammatory agents include any anti-inflammatory agent, including agents useful in therapies for inflammatory disorders, well-known to one of skill in the art. Non-limiting examples of anti-inflammatory agents include non-steroidal anti-inflammatory drugs (NSAIDs), steroidal anti-inflammatory drugs, anticholinergics (e.g., atropine sulfate, atropine methylnitrate, and ipratropium bromide (ATROVENT™)), β2-agonists (e.g., albuterol (VENTOLIN™ and PROVENTIL™), bitolterol (TORNALATE™), levalbuterol (XOPONEX™), metaproterenol (ALUPENT™), pirbuterol (MAXAIR™), terbutlaine (BRETHAIRE™ and BRETHINE™), albuterol (PROVENTIL™, REPETABS™, and VOLMAX™), formoterol (FORADIL AEROLIZER™), and salmeterol (SEREVENT™ and SEREVENT DISKUS™)), and methylxanthines (e.g., theophylline (UNIPHYL™, THEO-DUR™, SLO-BID™, AND TEHO-42™)). Examples of NSAIDs include, but are not limited to, aspirin, ibuprofen, celecoxib (CELEBREX™), diclofenac (VOLTAREN™), etodolac (LODINE™), fenoprofen (NALFON™), indomethacin (INDOCIN™), ketoralac (TORADOL™), oxaprozin (DAYPRO™), nabumentone (RELAFEN™), sulindac (CLINORIL™), tolmentin (TOLECTIN™), rofecoxib (VIOXX™), naproxen (ALEVE™, NAPROSYN™), ketoprofen (ACTRON™) and nabumetone (RELAFEN™). Such NSAIDs function by inhibiting a cyclooxygenase enzyme (e.g., COX-1 and/or COX-2). Examples of steroidal anti-inflammatory drugs include, but are not limited to, glucocorticoids, dexamethasone (DECADRON™), corticosteroids (e.g., methylprednisolone (MEDROL™)), cortisone, hydrocortisone, prednisone (PREDNISONE™ and DELTASONE™), prednisolone (PRELONE™ and PEDIAPRED™), triamcinolone, azulfidine, and inhibitors of eicosanoids (e.g., prostaglandins, thromboxanes, and leukotrienes. In other embodiments, the therapy(ies) used in accordance with the present application is not an anti-inflammatory agent.

In certain embodiments, the therapy(ies) used is an alkylating agent, a nitrosourea, an antimetabolite, and anthracyclin, a topoisomerase II inhibitor, or a mitotic inhibitor. Alkylating agents include, but are not limited to, busulfan, cisplatin, carboplatin, chlorambucil, cyclophosphamide, ifosfamide, decarbazine, mechlorethamine, melphalan, and themozolomide. Nitrosoureas include, but are not limited to carmustine (BCNU) and lomustine (CCNU). Antimetabolites include but are not limited to 5-fluorouracil, capecitabine, methotrexate, gemcitabine, cytarabine, and fludarabine. Anthracyclines include but are not limited to daunorubicin, doxorubicin, epirubicin, idarubicin, and mitoxantrone. Topoisomerase II inhibitors include, but are not limited to, topotecan, irinotecan, etoposide (VP-16), and teniposide. Mitotic inhibitors include, but are not limited to taxanes (paclitaxel, docetaxel), and the vinca alkaloids (vinblastine, vincristine, and vinorelbine).

In certain embodiments, the modified nanoparticles of the present application can be administered together with one or more antibiotic agents. In certain non-limiting embodiments, the antibiotic is a macrolide (e.g., tobramycin), a cephalosporin (e.g., cephalexin, cephradine, cefuroxime, cefprozil, cefaclor, cefixime or cefadroxil), a clarithromycin, an erythromycin, a penicillin (e.g., penicillin V) or a quinolone (e.g., ofloxacin, ciprofloxacin or norfloxacin), a tetracycline, a streptomycin, etc. In a particular embodiment, the antibiotic is active against Gram(+) and/or Gram(−) bacteria, e.g., Pseudomonas aeruginosa. Staphylococcus aureus, and the like.

In certain embodiments, modified nanoparticles are used in combination with topical agents that are contemplated to be selectably used for treatment of burns and wound healing. These topical agents can included, but are not limited to: albumin-based solutions, growth factors such as human recombinant epidermal growth factor, vascular endothelial growth factor, recombinant human basic fibroblast growth factor, keratocyte growth factor, platelet-derived growth factor, transforming growth factor beta, and nerve growth factor; anabolic hormones such as growth hormone and human insulin; any protease inhibitor such as nafamostat mesilate; any antibiotic compound at doses shown to safe and effective for human use such as a triple antibiotic (neomycin, polymyxin B, and bacitracin), neomycin, and mupirocin; and the gastric pentapeptide BPC 157.

In some embodiments, modified nanoparticle is used in combination with radiation therapy comprising the use of x-rays, gamma rays and other sources of radiation to destroy cancer stem cells and/or cancer cells. In specific embodiments, the radiation therapy is administered as external beam radiation or teletherapy, wherein the radiation is directed from a remote source. In other embodiments, the radiation therapy is administered as internal therapy or brachytherapy wherein a radioactive source is placed inside the body close to cancer stem cells, cancer cells and/or a tumor mass.

Currently available cancer therapies and their dosages, routes of administration and recommended usage are known in the art and have been described in such literature as the Physician's Desk Reference (60th ed., 2006). In accordance with the present application, the dosages and frequency of administration of chemotherapeutic agents are described supra.

### 6. EXAMPLES

### 6.1 Early Cytokine Response to Lipopolysaccharide (LPS)

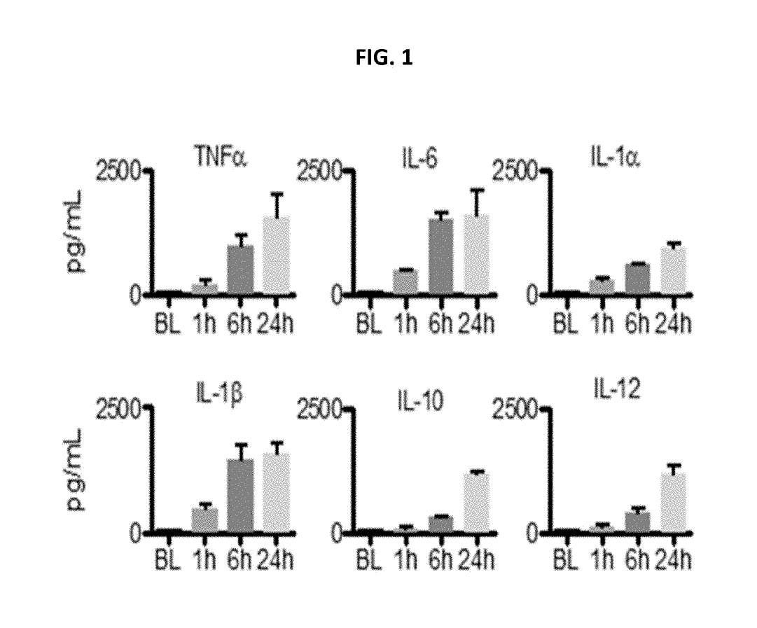

Cytokines in Balb/c mice after iv LPS (10 mg/kg) injection showed a significant increase in proinflammatory cytokines, as early as 1 hour. FIG. 1 shows that administration of NO (NO nanoparticles) early on after inoculation with LPS, prevents the progressive increase of inflammatory cytokines over time. Interestingly, proinflammatory cytokines increased at a faster rate (TNFα, IL-1β and IL-6) than repair cytokine IL-10 without NO supplementation.

This process is at the heart of many non-infectious conditions including, but not limited to: cardiovascular disease, neurological conditions (including various brain cancers such as glioblastoma, as well as neurodegenerative diseases such as Alzheimer's), diabetes, edema, and cancers (especially metastases). Thus, the use of sustained release NO nanoparticle or S-nitrosothiols (NACSNO), alone or in combination with other treatments, may serve to reduce fluid loss and tissue swelling in any of these diseases characterized by “leaky vasculature.”

This inflammatory process is also at the heart of many infectious conditions/diseases such as sepsis caused by bacterial or viral agents, and in particular, various hemorrhagic fevers including Filoviral infections such as Ebola and Marburg virus. The lethal consequences of many hemorrhagic fevers such as Ebola and Marburg arise from the onset of a massive inflammatory response occurring several days after the onset of the first symptoms. Thus, the use of sustained release NO nanoparticle or S-nitrosothiols (NACSNO), alone or in combination with other treatments, may serve to reduce fluid loss and tissue swelling in any of these diseases characterized by “leaky vasculature.”

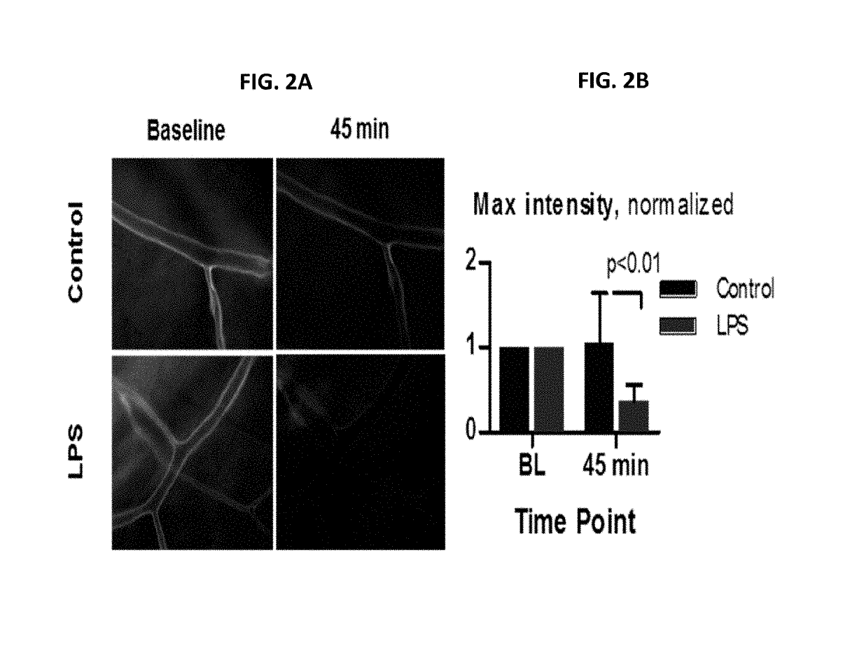

The endothelial lining of microvessels is structurally compromised during the onset of severe inflammation. The vascular endothelium is coated with glycocalyx, a fine layer of glycoproteins and glycosaminoglycans (GAG), with several roles in the maintenance of microvascular homeostasis, specifically cell-endothelium interactions, vascular permeability and signal mechanotransduction. Studies have pointed out the role of glycocalyx shredding on the beginning and progression of inflammatory diseases. FIGS. 2A-B show a rapid shredding of the glycocalyx, in a time scale similar to the reduction of functional capillary density and decrease capillary flow, during severe inflammation.

Endothelial glycocalyx shredding Balb/c mice were i.v. injected with 2 mg/kg of fluorescein isothiocyanate (FITC) conjugated lectin, which strongly binds to glycoproteins. FITC-conjugated lectin labeled the endothelial glycocalyx of microvessels. The same microvessels were followed for 3 hours after LPS infusion. These data illustrate that 45 mins after LPS injection, the glycocalyx has been shredded almost entirely (FIG. 2A, bottom panels) as compared to control animals. Infusion of NO nanoparticles decreased the changes in vascular permeability and prevented the rapid disruption of the vascular integrity. Changes in microvascular permeability to macromolecules occurs in a short time scale, consistent with the decrease in capillary perfusion. One of the hallmarks of severe inflammation is an increase in vascular permeability, mainly stimulated by the accumulation of proinflammatory cytokines. This increase in vascular permeability has been proposed to be the main cause of the hypotension during the shock, reduced perfusion and multi-organ failure. These results suggest that in small blood vessels (from capillaries to 100 μm blood vessels), the increase in vascular permeability occurs as early as 1 hour, and that NO supplementation with NO nanoparticles reduces and slows down the changes in vascular permeability. These data suggest that the increase in permeability might not be entirely mediated by cytokines, but also from an increase in intracellular Ca²⁺, from endoplasmic reticulum resulting from intracellular stress, that also function as intracellular signaling molecules underlying barrier failure via vascular endothelial (VE) cadherins.

### 6.2 Microvascular Permeability

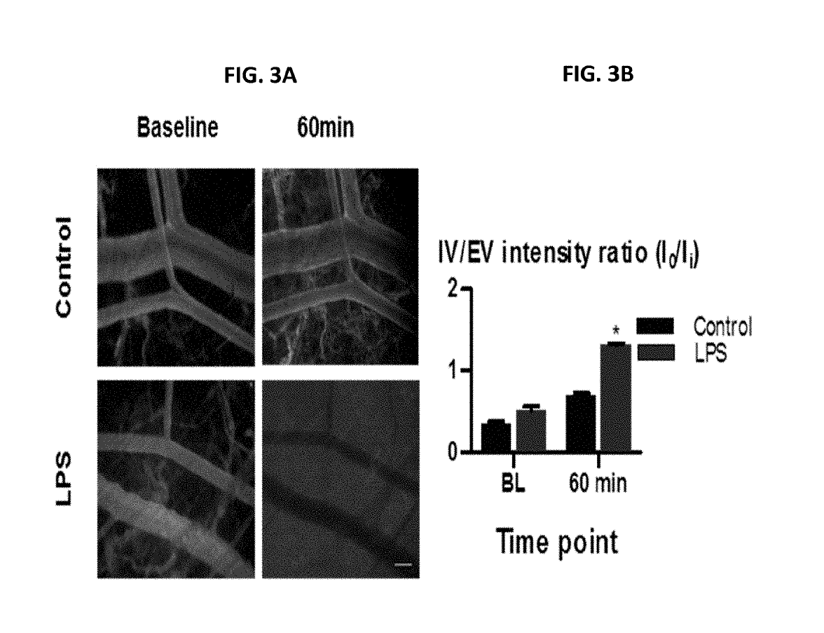

FITC conjugated dextran 70 kDa (FITC-dex) was injected through the tail vein in Balb/c mice, to quantify vascular endothelial permeability. Then, they received LPS (10 mg/kg) by i.v. injection. FIG. 3A shows that in the LPS animals (n=4), FITC-dex completely extravasated after 60 mins; whereas FITC-dex remained in the intravascular compartment in the control mice (n=4). The increase in vascular permeability explains the increase in capillary hematocrit (Hct) and the decrease in capillary blood flow. Increased vascular permeability, increase interstitial fluid pressure, reduced capillary pressure, and limited fluid exchange between compartments (Starling's law) are shown in FIG. 3B.

### 6.3 Control of Systemic Inflammation by Early NO Supplementation with NO-Releasing Nanoparticles

In this study, the effect of early treatment with nitric oxide (NO) supplementation with NO-releasing nanoparticles (NO-NPs) was evaluated in animal models. C57BL mice (8 weeks old; 2.2 g) were inoculated with 10 mg of LPS to induce systemic inflammation. The mice were then randomly separated into two experimental groups: the first group (n=6) to receive NO-NPs and the second group (n=6) to receive control nanoparticles (i.e., without NO). The mice then received 10 mg/kg of NO-NPs (or control NPs) intravenously 4 hours after LPS inoculation. A window is implanted in the animal that allows for monitoring blood flow.

The mice were studied at baseline (prior to LPS inoculation), 24 hours after LPS inoculation (early inflammation), and 48 hours after LPS inoculation (late inflammation). In particular, the macrophages of the mice were studied using flow cytometry and Mouse Oxidative Stress ELISA Strip (TNFα, TGFβ, MCP-1, IL-1α, IL-1β, IL-6, IL-10, IL-12). To avoid loss of surface markers, the lungs, spleens, and livers were minced and filtered four times through graded nylon filters, centrifuged at 1200 RPM for 5 minutes, and then re-suspended in erythrocyte lysis buffer. Cells were washed three times in nuclease-free PBS containing 2% bovine serum albumin. The cells were incubated with anti-mouse CD14/CD163 and CD11c/CD206 (BD Biosciences). Samples were washed and analyzed using flow cytometry. The survival proportions for both experimental groups was also evaluated over 72 hours following LPS inoculation.

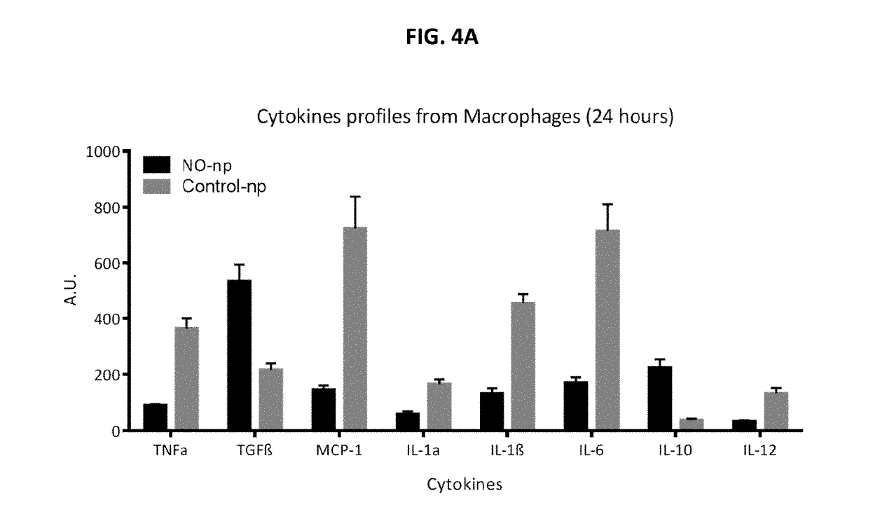

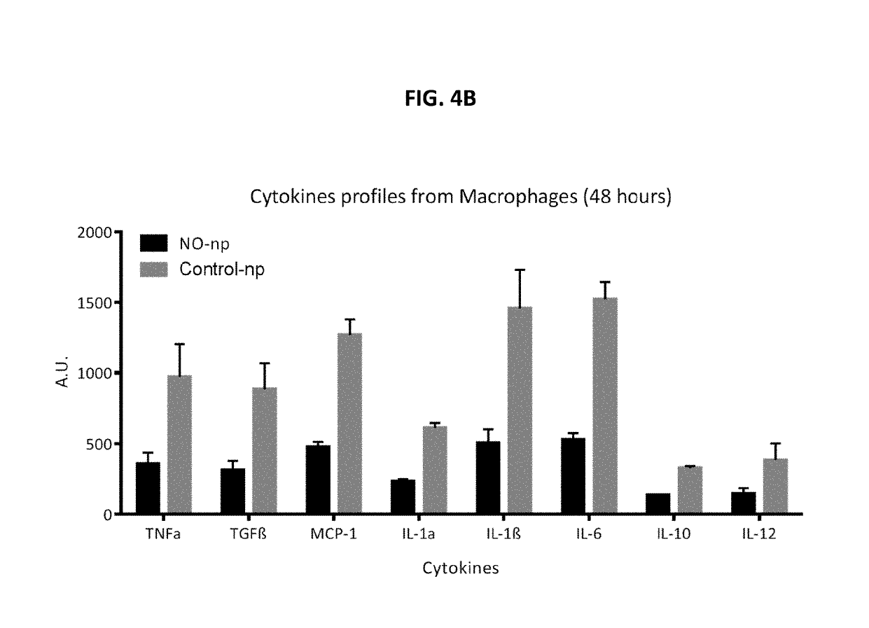

FIGS. 4A and 4B show the cytokine profiles from the macrophages for both experimental groups (NO-NPs and control NPs) at 24 hours and 48 hours. The results show that at 48 hours (FIG. 4B), there were lower levels of all cytokines for the NO-NPs group relative to the control NPs group. Further, at 48 hours, there were markedly lower levels of the proinflammatory cytokines (TNFα, IL-1β, IL-6) for the NO-NPs group relative to the control NPs group. These results suggest that treatment with NO-NPs results in decreased systemic inflammation.

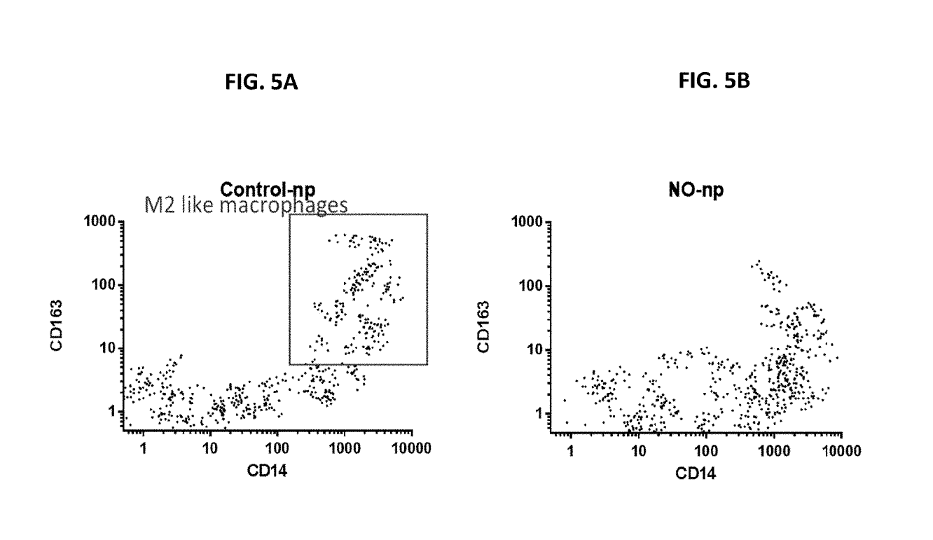

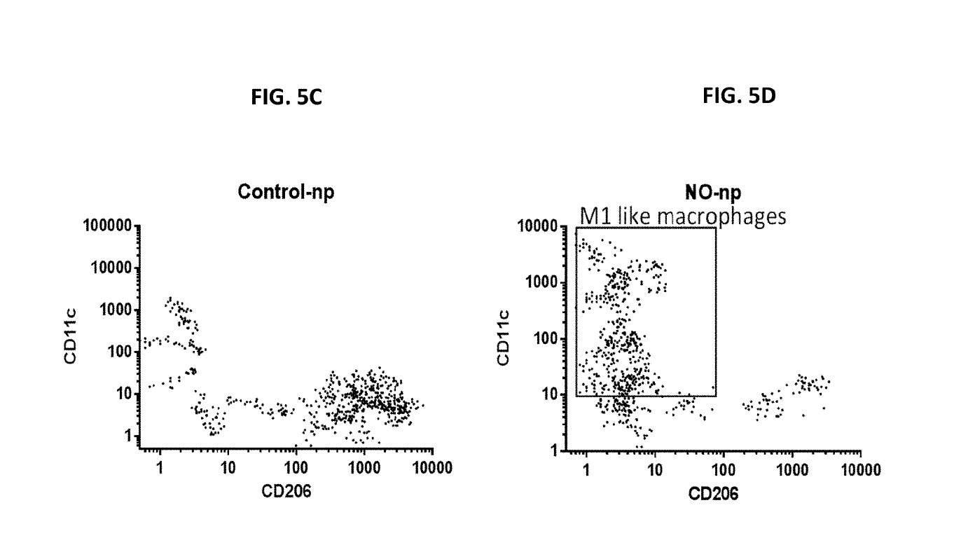

FIGS. 5A-D show the flow cytometry results for cells from both experiment groups incubated with anti-mouse CD14/CD163 (FIGS. 5A-B) and CD11c/CD206 (FIGS. 5C-D). The results show that Nonp treatment results in a change in macrophage population compared to the control. For the control, LPS induces an increase in the population of macrophages that are associated with causing the potentially lethal shock that results from vascular inflammatory cascades. Treatment with NOnp not only inhibits the build up of the “shock” associated macrophages but also stimulates the build up the macrophages associated with repair.

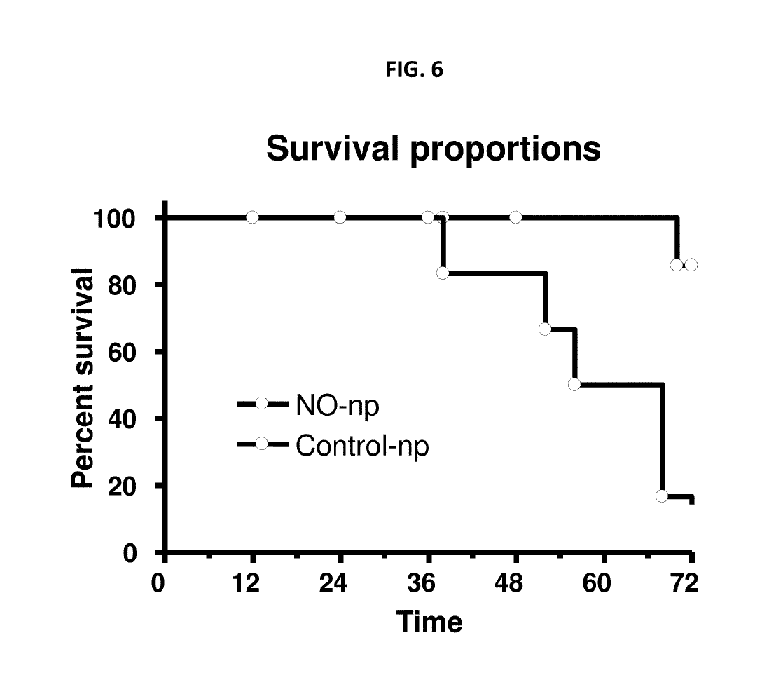

FIG. 6 shows the survival proportions for both experimental groups. The results showed that treatment with NO-NPs resulted in marked increase in survival at 72 hours after LPS administration relative to treatment with control NPs.

### 6.4 Reversal of Systemic Inflammation by NO Supplementation with NO-Releasing Nanoparticles

In this study, the effect of treatment with nitric oxide (NO) supplementation via NO-releasing nanoparticles (NO-NPs) was evaluated in animal models. C57BL mice (8 weeks old; 2.2 g) were inoculated with 10 mg of LPS to induce systemic inflammation. The mice were then randomly separated into two experimental groups: the first group (n=5) to receive NO-NPs and the second group (n=5) to receive control nanoparticles (i.e., without NO). The mice then received a 10 mg/kg infusion of NO-NPs (or control NPs) intravenously 24 hours after LPS inoculation.

The mice were studied at baseline (prior to LPS inoculation), 24 hours after LPS inoculation (at the time of nanoparticle infusion), 36 hours after LPS inoculation (early inflammation), and 48 hours after LPS inoculation (late inflammation). Cytokine profiles for both experimental groups were determined at baseline, 24 hours after LPS inoculation, and 48 hours for both experimental groups using Mouse Oxidative Stress ELISA Strip (TNFα, TGFβ, MCP-1, IL-1α, IL-1β, IL-6, IL-10, IL-12). The survival proportions for both experimental groups was also evaluated over 72 hours following LPS inoculation.

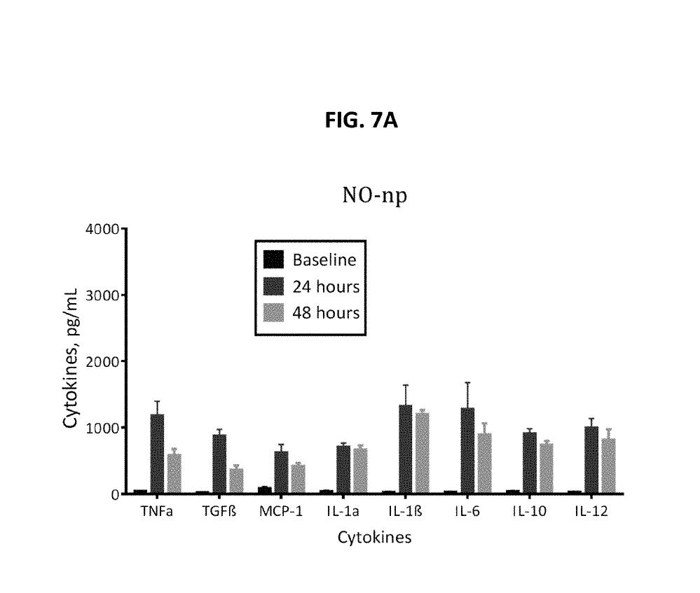

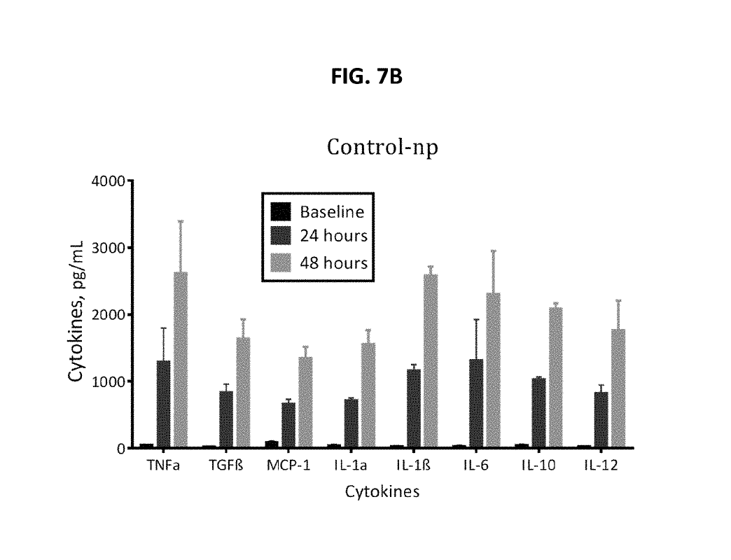

FIG. 7A shows the cytokine profile for the NO-NPs experimental group at baseline, 24 hours and 48 hours after LPS inoculation, while FIG. 7B shows the cytokine profile for the control NPs experimental group at baseline, 24 hours and 48 hours after LPS inoculation. The results for the NO-NPs experimental group show that cytokines levels rise following LPS inoculation (at time 0 hours) up through 24 hours (time of NO-NPs infusion). However, the results at 48 hours show a marked decrease in cytokine levels relative to levels at 24 hours (FIG. 7A). In contrast, for the control-NPs treated mice, cytokine levels steadily rose from baseline to 48 hours following LPS inoculation. The results also show that the levels of proinflammatory cytokines TNFα, IL-1β, IL-6) were markedly lower for the NO-NPs group at 48 hours relative to the control-NP group. These results suggest that infusion with NO-NPs results in the reversal of systematic inflammation.

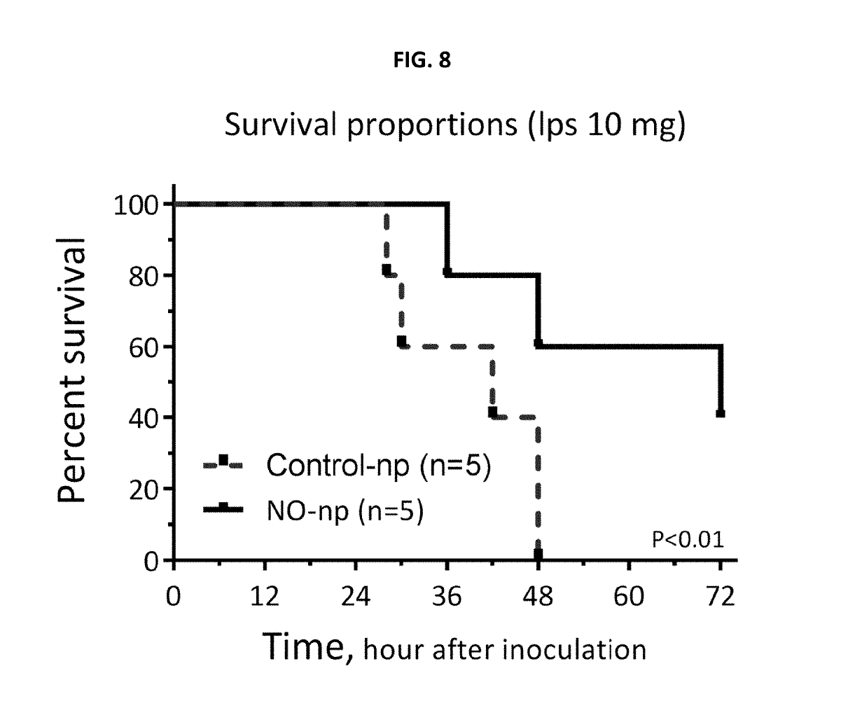

FIG. 8 shows the survival proportions for both experimental groups. These results showed that treatment with NO-NPs resulted in a significant increase in survival at 72 hours after LPS administration relative to treatment with control NPs (p<0.01).

### 6.5 Treatment with Curcumin-Selenium Nanoparticles in Subjects with Endotoxemia

In this study, the effect of treatment with curcumin-selenium nanoparticles was evaluated in animal models with endotoxemia. In particular, C57BL mice were first infused with 10 mg/kg of LPS (Lipopolysaccharides from E. coli serotype 0128:B12, Sigma Aldrich St. Louis, Mo.). The mice were then separated into 5 treatment groups: 1) no treatment; 2) control NP 10 mg/kg; 3) curcumin 10 mg/kg; 4) curcumin-NP 10 mg/kg (dose calculated based on curcumin concentration); and 5) curcumin-selenium-NP 10 mg/kg (dose calculated based on curcumin concentration). Treatments were administered 30 minutes after LPS infusion. No additional fluid therapies were applied, and food and water was available during observation time points.

Cytokine profiles for all experimental groups were determined at baseline, and at 1, 6, and 24 hours after LPS infusion using Mouse Oxidative Stress ELISA Strip (TNFα, TGFβ, MCP-1, IL-1α, IL-1β, IL-6, IL-10, IL-12). Vascular integrity for all treatment groups was also evaluated at baseline and 2 hours after LPS infusion. Additionally, other end points related to systemic and microvascular function (i.e., heart rate, arteriolar diameter, arteriolar blood flow, and functional capillary density) were evaluated for 4 of the treatment groups (no treatment, control NP 10, curcumin-NP 10 mg/kg, and curcumin-selenium-NP 10 mg/kg treatment groups) at baseline, and 2, 4, 6, 12, and 24 hours after LPS infusion.

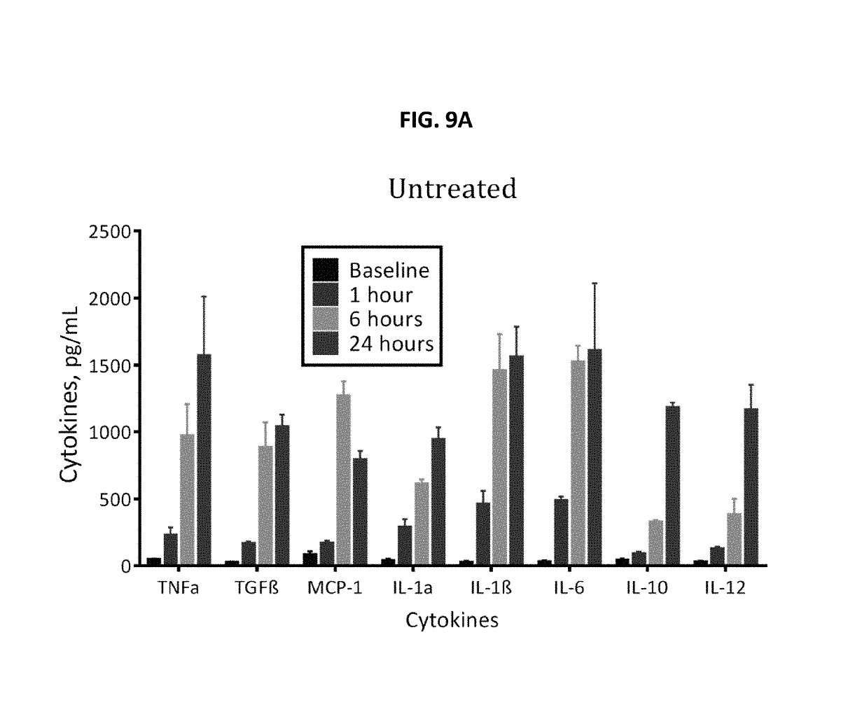

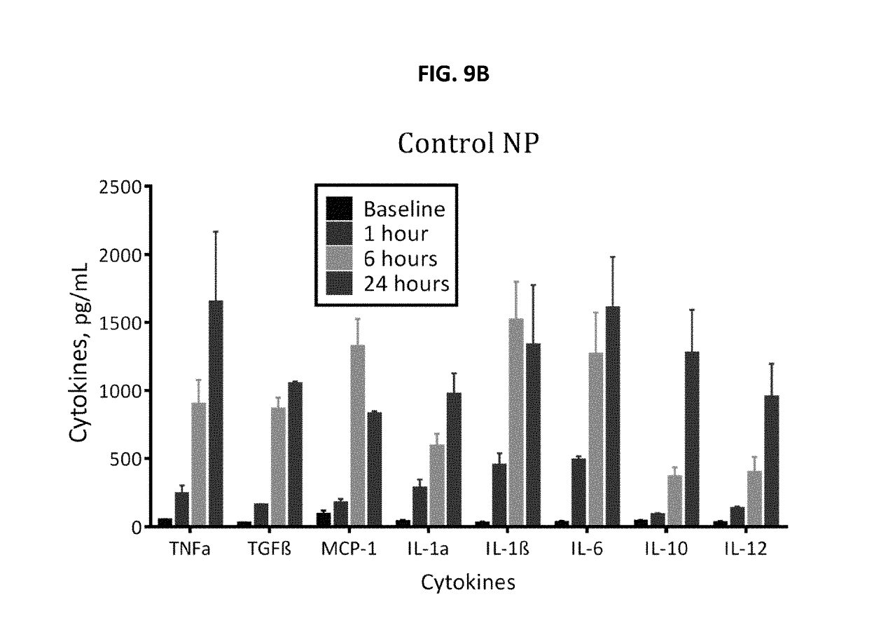

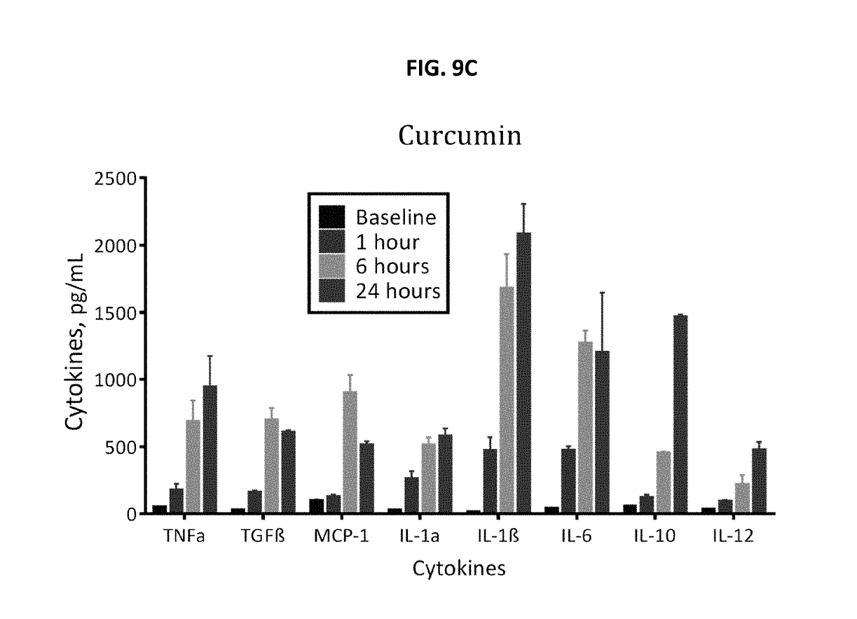

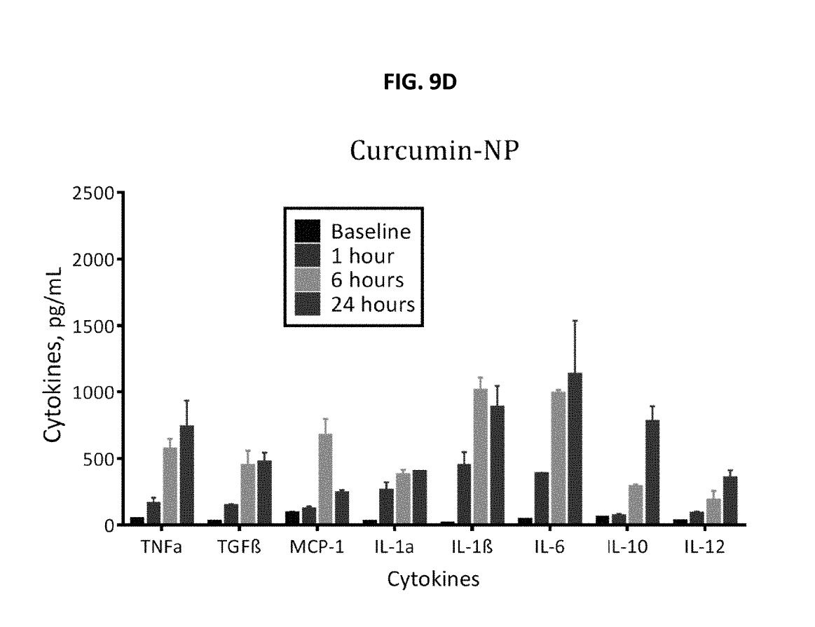

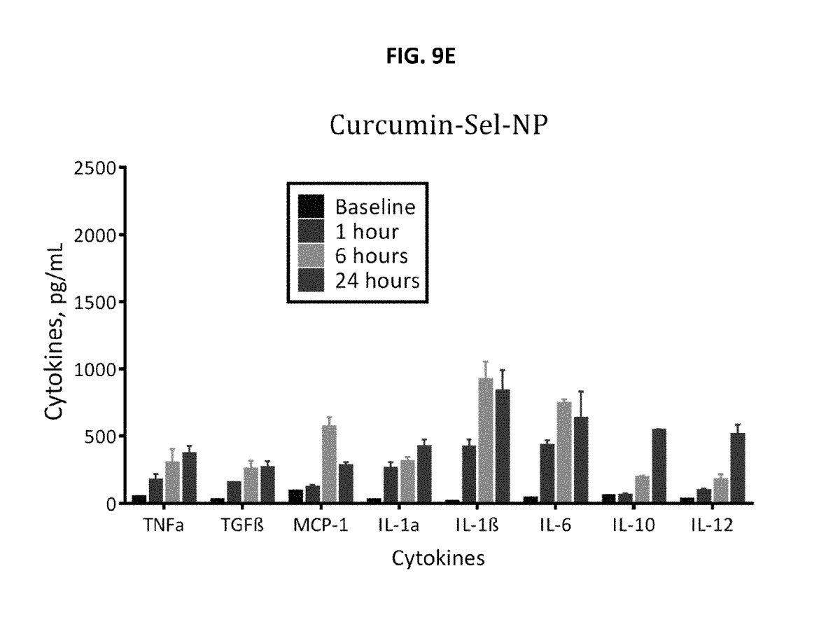

FIGS. 9A-E show the cytokine profile for all experimental groups at baseline, and at 1, 6, and 24 hours after LPS infusion. The results show that treatment with curcumin-selenium nanoparticles (curcumin-sel-NP) resulted in lower levels of all cytokines (TNFα, TGFβ, MCP-1, IL-1α, IL-β, IL-6, IL-10, IL-12) at 24 hours after LPS infusion relative to the other treatment groups. Further, the results show that treatment with curcumin-selenium nanoparticles (curcumin-sel-NP) resulted in markedly lower levels of proinflammatory cytokines (TNFα, IL-1β, IL-6) at 24 hours after LPS infusion relative to the other treatment groups. These results suggest that treatment with curcumin-sel-NPs results in a decrease in systemic inflammation.

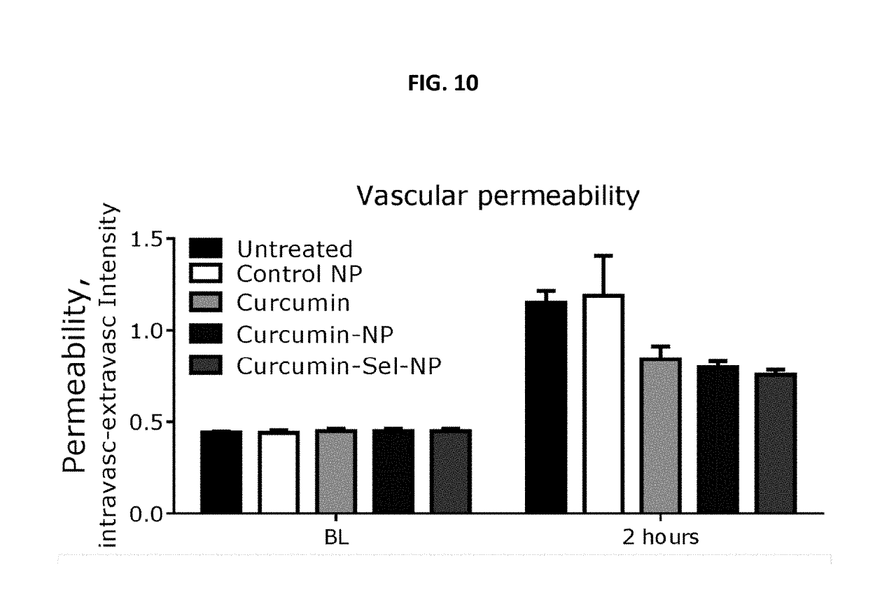

FIG. 10 compares the vascular permeability (intravascular-extravascular intensity) for all treatment groups at baseline and 2 hours after LPS infusion. All groups showed increased vascular permeability at 2 hours relative to baseline. However, the curcumin, curcumin-NP, and curcumin-sel-NP treatment groups showed markedly less vascular permeability at 2 hours as compared with the untreated and control NP treatment groups, with the curcumin-sel-NP treatment group showing the lowest vascular permeability. These results suggest that treatment with curcumin with or without selenium can result in an immediate reduction in vascular permeability.

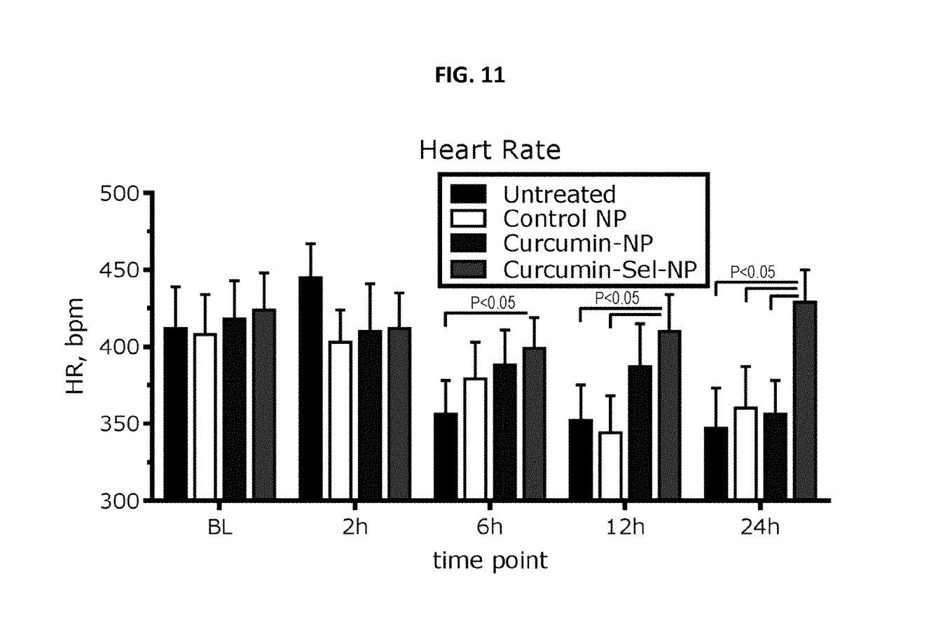

FIG. 11 compares the heart rates (HR) for 4 treatment groups at baseline, and at 2, 6, 12, and 24 hours after LPS infusion. The curcumin-sel-NP treatment resulted in the maintenance HR at near baseline levels at 2, 6, 12, and 24 hours. Further, the curcumin-sel-NP treatment group showed significant increases at 6, 12, and 24 hours after LPS infusion relative to other treatment groups. In particular, at 6 hours, curcumin-sel-NP treatment group showed significant increases in HR relative to the untreated group (p<0.05). At 12 hours, the curcumin-sel-NP treatment group showed significant increases in HR relative to both the control-NP group and the untreated group (p<0.05). At 24 hours, the curcumin-sel-NP treatment group showed significant increases in HR relative to the control-NP group, the untreated group, and the curcumin-NP group (p<0.05).

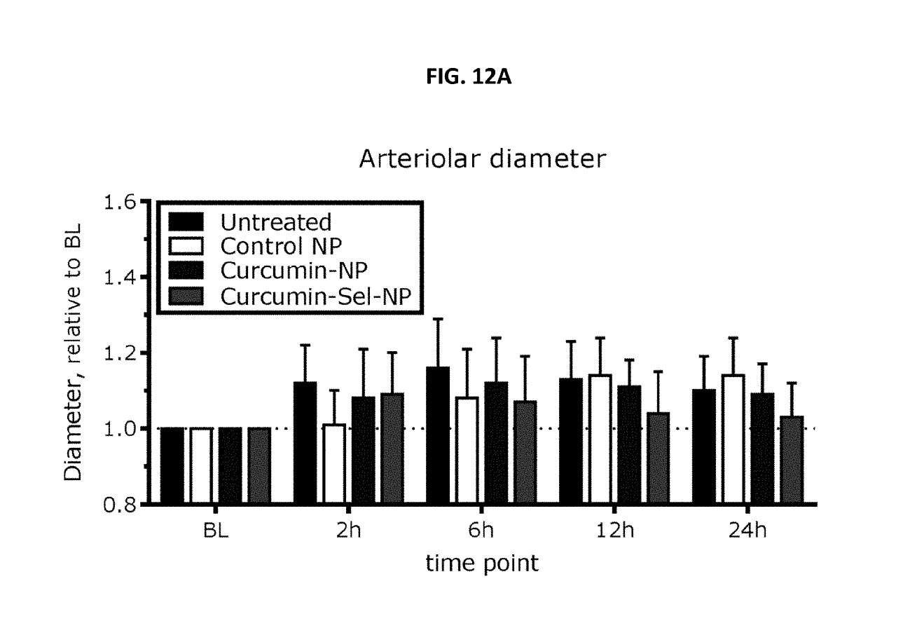

FIGS. 12A-B compare the microcirculation hemodynamics, specifically arteriolar diameter (FIG. 12A) and arteriolar blood flow (FIG. 12B), for 4 treatment groups at baseline, and at 2, 6, 12, and 24 hours after LPS infusion. As shown in FIG. 12A, treatment with curcumin-sel-NP returned arteriolar diameter to nearly baseline levels at 24 hours after LPS infusion, while the other treatment groups displayed elevated arteriolar diameters at 24 hours. Similarly, FIG. 12B shows that treatment with curcumin-sel-NP maintained arteriolar blood flow near baseline levels following after LPS infusion. Additionally, the curcumin-sel-NP treatment group displayed significantly higher blood flow at 6, 12, and 24 hours as compared with the untreated, control-NP, and curcumin-NP treatment groups (p<0.05). These results suggest that treatment with curcumin-sel-NP can maintain arteriolar blood flow in subjects with systemic inflammation.

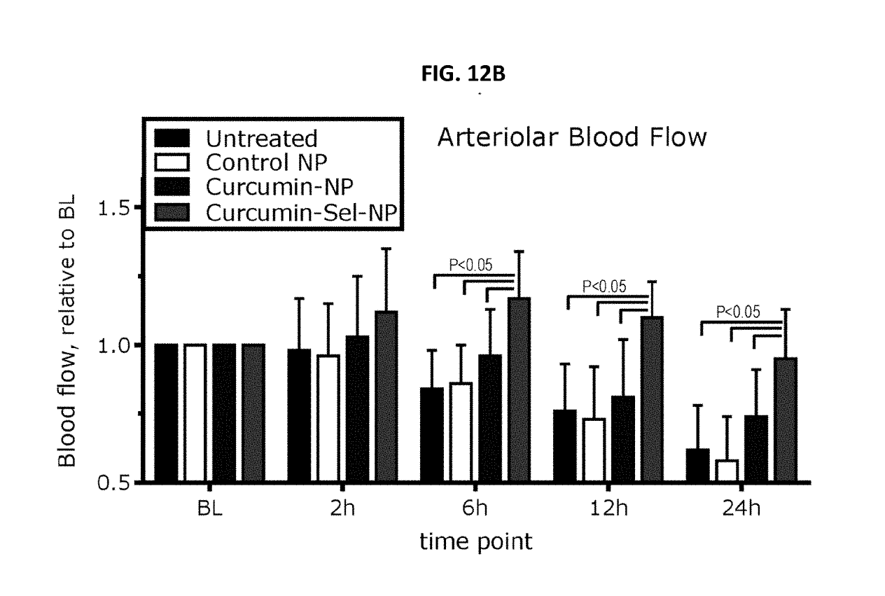

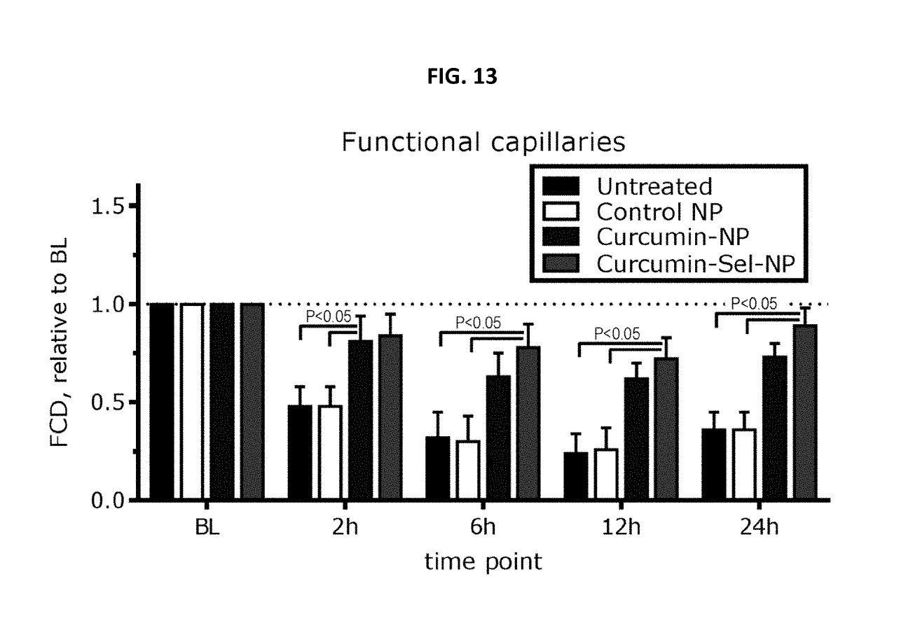

FIG. 13 compares the functional capillary density (FCD) for 4 treatment groups at baseline, and at 2, 6, 12, and 24 hours after LPS infusion. As shown in FIG. 13, treatment with curcumin-sel-NP returned FCD to nearly baseline levels at 24 hours after LPS infusion. Further, the curcumin-sel-NP treatment group displayed significantly higher FCD relative to untreated and control-NP treatment at, 6, 12, and 24 hours after LPS infusion (p<0.05).

In accordance with the present invention, there may be numerous tools and techniques within the skill of the art, such as those commonly used in molecular immunology, cellular immunology, pharmacology, and microbiology. See, e.g., Sambrook et al. (2001) Molecular Cloning: A Laboratory Manual. 3rd ed. Cold Spring Harbor Laboratory Press: Cold Spring Harbor, N.Y.; Ausubel et al. eds. (2005) Current Protocols in Molecular Biology. John Wiley and Sons. Inc.: Hoboken, N.J.; Bonifacino et al. eds. (2005) Current Protocols in Cell Biology. John Wiley and Sons, Inc.: Hoboken, N.J.; Coligan et al. eds. (2005) Current Protocols in Immunology, John Wiley and Sons, Inc.: Hoboken, N.J.; Coico et al. eds. (2005) Current Protocols in Microbiology, John Wiley and Sons, Inc.: Hoboken, N.J.; Coligan et al. eds. (2005) Current Protocols in Protein Science, John Wiley and Sons, Inc.: Hoboken, N.J.; and Enna et al. eds. (2005) Current Protocols in Pharmacology, John Wiley and Sons, Inc.: Hoboken, N.J.

In accordance with the present invention there may be employed conventional molecular biology, microbiology, protein expression and purification, antibody, and recombinant DNA techniques within the skill of the art. Such techniques are explained fully in the literature. See, e.g., Sambrook, Fritsch and Maniatis. Molecular Cloning: A Laboratory Manual, Second Edition (Cold Spring Harbor Laboratory Press, New York: 1989); DNA Cloning: A Practical Approach, Volumes I and II (Glover ed.: 1985); Oligonucleotide Synthesis (Gait ed.: 1984); Nucleic Acid Hybridization (Hames & Higgins eds.: 1985); Transcription And Translation (Hames & Higgins, eds.: 1984); Animal Cell Culture (Freshney, ed.: 1986); Immobilized Cells And Enzymes (IRL Press: 1986); Perbal, A Practical Guide To Molecular Cloning (1984); Ausubel et al., eds. Current Protocols in Molecular Biology, (John Wiley & Sons, Inc.: 1994); and Harlow and Lane. Antibodies: A Laboratory Manual (Cold Spring Harbor Laboratory Press: 1988).

All publications mentioned herein are hereby incorporated in their entireties into the subject application. Where there is an apparent conflict between a term as used herein and the same term as used in a publication incorporated by reference herein, the present specification is understood to provide the controlling definition.

While the foregoing invention has been described in some detail for purposes of clarity and understanding, it will be appreciated by one skilled in the art from a reading of the disclosure that various changes in form and detail can be made without departing from the true scope of the invention in the appended claims.

### REFERENCES

Zacharia I. G., Deen W. M. Diffusivity and solubility of nitric oxide in water and saline. Ann Biomed Eng. 2005; 33:214-222.
Moncada S., Palmer M. J, Higgs E. A. Nitric Oxide: Physiology, pathophysiology, and pharmacology. Pharmacol Rev. 1991; 43:109-134.
Busse R., Fleming I. Pulsatile stretch and shear stress: physical stimuli determining the production of endothelium-derived relaxing factors. J Vasc Res. 1998; 35:73-84.
Lancaster J. R., Jr. A tutorial on the diffusibility and reactivity of free nitric oxide. Nitric Oxide. 1997; 1:18-30.
Ichinose F., Roberts J. D., Jr, Zapol W. M. Inhaled nitric oxide: a selective pulmonary vasodilator: current uses and therapeutic potential. Circulation. 2004; 109:3106-3111.
Zapol W. M. Inhaled nitric oxide. Acta Anaesthesiol Scand Suppl. 1996; 109:81-83.
Homer K., Wanstall J. In vitro comparison of two NONOates (novel nitric oxide donors) on rat pulmonary arteries. Eur J Pharmacol. 1998; 356:49-57.
Friedman, A. J., Han, G., Navati, M. S., Chacko, M., Gunther, L., Alfieri, A., and Friedman,
J. M. (2008) Sustained release nitric oxide releasing nanoparticles: characterization of a novel delivery platform based on nitrite containing hydrogel/glass composites. Nitric Oxide 19, 12-20.
Nachuraju, P., Friedman. A. J., Friedman, J. M., and Cabrales, P. (2011) Exogenous nitric oxide prevents cardiovascular collapse during hemorrhagic shock. Resuscitation 82, 607-613.
Cabrales, P., Han, G., Nacharaju, P., Friedman, A. J., and Friedman, J. M. (2011) Reversal of hemoglobin-induced vasoconstriction with sustained release of nitric oxide. Am J Physiol Heart Circ Physiol 300, H49-56.
Han, G., Tar, M., Kuppam, D. S., Friedman, A., Melman, A., Friedman, J., and Davies, K. P.
(2010) Nanoparticles as a novel delivery vehicle for therapeutics targeting erectile dysfunction. J Sex Med 7, 224-233.
Cabrales, P., Han, G., Roche, C., Nacharaju, P., Friedman, A. J., and Friedman, J. M. (2010) Sustained release nitric oxide from long-lived circulating nanoparticles. Free Radic Biol Med 49, 530-538.
Nacharaju, P., Tuckman-Vernon, C., Maier, K. E., Chouake, J., Friedman, A., Cabrales, P., and Friedman, J. M. (2012) A nanoparticle delivery vehicle for S-nitroso-N-acetyl cysteine: Sustained vascular response. Nitric Oxide 27, 150-160.

1. A method for early treatment of systemic inflammation in a subject comprising administering a therapeutically effective amount of curcumin and selenium-loaded nanoparticles to the subject within 24 hours of the onset of systemic inflammation, wherein the curcumin and selenium-loaded nanoparticles comprise a matrix of chitosan, polyethylene glycol (PEG) and tetramethoxysilane (TMOS) encapsulating the curcumin and selenium.

2. The method of claim 1, wherein the systemic inflammation is caused by endotoxemia.

3. The method of claim 1, wherein systemic inflammation in the subject is characterized by an increase in cytokines.

4. The method of claim 1, wherein the systemic inflammation is caused by a Filovirus.

5. The method of claim 4, wherein the Filovirus is an Ebola virus.

6. The method of claim 4, wherein the Filovirus is a Marburg virus.

7. The method of claim 4, wherein the curcumin and selenium-loaded nanoparticles are administered in combination with one or more antiviral treatments.

8. The method of claim 1, wherein the curcumin and selenium-loaded nanoparticles are administered in combination with one or more anti-inflammatory treatments.

9. The method of claim 1, wherein the administering of the curcumin and selenium-loaded nanoparticles results in a 20-30% increase in heart rate in the subject.

10. The method of claim 1, wherein the administering of the curcumin and selenium-loaded nanoparticles results in a 10-15% decrease in arteriolar diameter in the subject.

11. The method of claim 1, wherein the administering of the curcumin and selenium-loaded nanoparticles results in a 50-60% increase in arteriolar blood flow in the subject.

12. The method of claim 1, wherein the administering of the curcumin and selenium-loaded nanoparticles results in a 100-200% increase in functional capillary density in the subject.

13. The method of claim 1, wherein the administration of the curcumin and selenium-loaded nanoparticles results in a reduction in one or more cytokines in the subject, wherein the cytokines are selected from the group consisting of TNFα, TGFβ, MCP-1, IL-1α, IL-1β, IL-4, IL-6, IL-10, and IL-1.

14. The method of claim 1, wherein the administration of the curcumin and selenium-loaded nanoparticles results in a reduction in one or more proinflammatory cytokines in the subject, wherein the proinflammatory cytokines are selected from the group consisting of TNFα, IL-1β, and IL-6.

15. The method of claim 14, wherein the proinflammatory cytokines are reduced by 50-60%.

16. The method of claim 1, wherein the administering of the curcumin and selenium-loaded nanoparticles results in a 30-40% decrease in vascular permeability in the subject.

17. The method of claim 1, wherein the curcumin and selenium-loaded nanoparticles are administered by incorporation into an intravenous (IV) infusion, or by transmucosal systemic delivery.

18. A method of treating systemic inflammation in a subject comprising administering a therapeutically effective amount of curcumin and selenium-loaded nanoparticles to the subject, wherein the curcumin and selenium-loaded nanoparticles comprise a matrix of chitosan, polyethylene glycol (PEG) and tetramethoxysilane (TMOS) encapsulating the curcumin and selenium.

19. The method of claim 18, wherein the systemic inflammation is caused by endotoxemia.

20. The method of claim 18, wherein the curcumin and selenium-loaded nanoparticles are administered prior to onset of symptoms of systemic inflammation.

21-66. (canceled)
